(19) United States
(12) Patent Application Publication
(10) Pub. No.: US 2020/0248156 A1
(43) Pub. Date: Aug. 6, 2020

(54) Targetable 3`-Overhang Nuclease Fusion Proteins

(71) Applicant: The General Hospital Corporation, Boston, MA (US)

(72) Inventors: J. Keith Joung, Winchester, MA (US); Rebecca Tayler Cottman, Cambridge, MA (US)

(21) Appl. No.: 16/779,327

(22) Filed: Jan. 31, 2020

Related U.S. Application Data
(60) Provisional application No. 62/800,000, filed on Feb. 1, 2019.
(60) Provisional application No. 62/908,963, filed on Oct. 1, 2019.

(51) Int. Cl. C12N 9/22; C12N 15/63

(52) U.S. Cl. CPC C12N 9/22; C07K 2319/00; C12N 2800/80; C12N 15/63

(57) ABSTRACT

Described herein are zinc finger and dCas9 nuclease fusion proteins and methods of using the same for enhancing repair frequencies at the site of a nuclease-induced double strand breaks (DSB) for use in genome editing.

### CROSS-REFERENCE TO RELATED APPLICATIONS

This application claims the benefit of U.S. Provisional Application Ser. No. 62/800,000, filed on Feb. 1, 2019, and U.S. Provisional Application Ser. No. 62/908,963, filed on Oct. 1, 2019. The entire contents of the foregoing are incorporated herein by reference.

### FEDERALLY SPONSORED RESEARCH OR DEVELOPMENT

This invention was made with government support under Grant No. GM118158 awarded by the National Institutes of Health. The government has certain rights in the invention.

### TECHNICAL FIELD

This invention relates, at least in part, to targetable 3′-overhang nucleases and methods of use thereof.

### BACKGROUND

Double strand breaks (DSBs) induced by genome-editing nucleases can be efficiently repaired by non-homologous end-joining (NHEJ) (or in some cases, an alternative NHEJ repair pathway known as microhomology-mediated end-joining or MMEJ), resulting in the efficient introduction of variable-length insertion or deletions (indels); alternatively, DSBs can also be repaired by homology-directed repair (HDR) with a homologous double-stranded or single-stranded DNA bearing a sequence alteration of interest to create precise changes (commonly referred to as the “donor template”). In most eukaryotes, and especially in human cells, NHEJ is the favored repair pathway at DSBs and therefore, indels are generally introduced more efficiently than more precise HDR-mediated changes. Thus, a major challenge for the genome editing field is promoting the efficiency of HDR-mediated repair events over variable-length NHEJ-mediated indels at nuclease-induced DSBs. Improving the efficiency of HDR will enable the unlocking of a much broader range of research applications as well as widen the number of gene-based diseases that might be treated using genome-editing nucleases.

Although several strategies have been proposed to improve the efficiency of nuclease-induced HDR, each of these approaches has limitations. Small molecules that inhibit NHEJ-specific factors (e.g., Scr7, which inhibits DNA Ligase IV) have been suggested as a strategy to increase rates of HDR, but these reagents are toxic, rendering them impractical for potential therapeutic applications (Maruyama, T. et al., Nature Biotechnology (2015); Shrivastav, M. et al. Cell Research (2007)). It has also been difficult to replicate the effects of Scr7 as some have shown it does not actually inhibit ligase IV (Greco, George E. et al., DNA Repair (2016). Other groups have found that they could slightly improve the rates of HDR by 2-fold by synchronizing in the M stage of the cell cycle before treating with nucleases (Lin, S., et al. eLife (2014)) but this process is also generally very toxic to cells making it an impractical approach for application in vivo. Modest improvements in HDR efficiency have also been reported by altering the extent of symmetry in the donor template around the DSB but it is unclear how generalizable even this modest effect is across different genes and cell types (Richardson, C., et al., Nature Biotechnology (2015)); Liang, Xiquan., et al. Journal of Biotechnology (2016)).

### SUMMARY

An effective technique for enhancing HDR frequencies at the site of a nuclease-induced DSB would be highly desirable for genome editing.

It has now been determined that fusion proteins comprising a DNA-targeting domain (e.g., an RNA-guided catalytically inactive Cas9 nuclease or an engineered zinc finger array) and a nuclease domain that generates 3′ overhang double strand breaks can enhance repair frequencies (e.g., HDR, NHEJ, MMEJ) at the site of the break and can be used to improve the efficiency of genome editing.

Other features and advantages of the invention will be apparent from the Detailed Description, and from the claims. Thus, other aspects of the invention are described in the following disclosure and are within the ambit of the invention.

In one aspect, the present disclosure relates to a DNA-binding domain (DBD) nuclease fusion protein including: (a) a dimerization-dependent nuclease domain, where the domain generates 3′ overhang double strand breaks in DNA; and (b) a DNA-binding domain (DBD), where the dimerization-dependent nuclease domain is a Type IIS restriction enzyme nuclease domain, optionally an AcuI nuclease domain.

In one embodiment, the dimerization-dependent nuclease domain is linked to the DBD with an amino acid linker. In one embodiment, the amino acid linker includes the amino acid sequence of SEQ ID NO:2 or SEQ ID NO:3. In another embodiment, the amino acid linker is an XTEN linker. In one embodiment, the DBD is a zinc finger array, a catalytically inactive Cas9 (dCas9) domain, or a TALE domain.

In one embodiment, he nuclease domain includes an AcuI nuclease or an isoschizomer of AcuI nuclease (e.g., Eco57I nuclease)

In one embodiment, the nuclease domain is an AcuI nuclease that includes an amino acid sequence that has at least 80%, at least 85%, at least 90%, or at least 95% sequence identity to the amino acid sequence of SEQ ID NO: 5.

In one embodiment, the amino acid domain is an AcuI nuclease domain that includes an amino acid sequence that has at least 80%, at least 85%, at least 90%, or at least 95% sequence identity to the amino acid sequence of SEQ ID NO: 4.

In one embodiment, the AcuI nuclease domain contains H3S, H5S, K6S, K11S, R14S, N15D, N19D, R20S, K21S, N25D, R27S, N29D, R34S, K50S, N51D, K52S, K55S, N58D, R60S, K69S, H75S, K77S, K78S, R84S, R89S, K90S, K96S, K97S, H101S, N106D, K110S, Q111E, R113S, R114S, K120S, K122S, N128D, K140S, N148D, K149S, R151S, K153S, K154S, H156S, H163S, R173S, N180D, K183S, N190D, K191S, N193D, H194S, K203S, Q204E, N206D, R209S, K218S, Q220E, Q224E, N226D, or N229D substitution mutation, or any combination thereof.

In one embodiment, the nuclease domain is fused to an amino-terminal end of the DBD. In another embodiment, the nuclease domain is fused to a carboxyl-terminal end of the DBD.

In one aspect, the present disclosure relates to a DBD nuclease fusion protein dimer complex including two monomer fusion proteins, where each monomer is any of the fusion proteins described herein.

In one embodiment, each of the DBD of the two monomer fusion proteins is a dCas9 domain, and the dimer complex binds to a target site in a PAM-out orientation.

In one aspect, the present disclosure relates to a method of copying, incorporating, and/or inserting a nucleic acid sequence from an exogenous donor template into a nuclease target site of a genomic locus of a cell, the method including providing an exogenous donor template and a nucleic acid sequence encoding any of the DBD nuclease fusion proteins described herein to the nucleus of a cell, where the exogenous donor template includes sequences homologous to sequences within the nuclease target site of the genomic locus, and where the DBD nuclease fusion protein binds to the nuclease target site and generates a 3′ overhang double strand break within the nuclease target site to induce homology-directed repair between the exogenous donor template sequences and the sequences surrounding the break, thereby copying, incorporating, and/or inserting the nucleic acid sequence from the exogenous donor template into the nuclease target site of the genomic locus of the cell.

In one embodiment, the copied, incorporated, or inserted nucleic acid sequence replaces or corrects a mutated sequence within the nuclease target site of the genomic locus.

In one embodiment, the copied, incorporated, or inserted nucleic acid sequence inhibits or activates expression of a gene within or adjacent to the nuclease target site of the genomic locus.

In one aspect, the present disclosure relates to a method of copying, incorporating, and/or inserting a nucleic acid sequence from an exogenous donor template into a dCas9 target site of a genomic locus of a cell, the method including providing an exogenous donor template and a nucleic acid sequence encoding any of the dCas9 nuclease fusion proteins described herein, and one or more dCas9-associated guide RNAs to the nucleus of a cell, where the exogenous donor template includes sequences homologous to sequences within the dCas9 target site of the genomic locus, and where the dCas9 nuclease fusion protein forms a complex with one or more guide RNAs, and the complex binds to the dCas9 target site to generates a 3′ overhang double strand break within the dCas9 target site to induce homology-directed repair between the exogenous donor template sequences and the sequences surrounding the break, thereby copying, incorporating, and/or inserting the nucleic acid sequence from the exogenous donor template into the dCas9 target site of the genomic locus of the cell.

In one aspect, the present disclosure relates to a method of copying, incorporating, and/or inserting a nucleic acid sequence from an exogenous donor template into a nuclease target site of a genomic locus of a cell, the method including providing an exogenous donor template and any of the zinc finger nuclease fusion proteins described herein to the nucleus of a cell, where the exogenous donor template includes sequences homologous to sequences within the nuclease target site of the genomic locus, and where the zinc finger nuclease fusion protein binds to the nuclease target site and generates a 3′ overhang double strand break within the nuclease target site to induce homology-directed repair between the exogenous donor template sequences and the sequences surrounding the break, thereby copying, incorporating, and/or inserting the nucleic acid sequence from the exogenous donor template into the nuclease target site of the genomic locus of the cell.

In one aspect, the present disclosure relates to a method of copying, incorporating, and/or inserting a nucleic acid sequence from an exogenous donor template into a dCas9 target site of a genomic locus of a cell, the method including providing an exogenous donor template, a dCas9 nuclease fusion protein, and one or more dCas9-associated guide RNAs to the nucleus of a cell, where the exogenous donor template includes sequences homologous to sequences within the dCas9 target site of the genomic locus, and where the dCas9 nuclease fusion protein is in a complex with one or more guide RNA(s), and the complex binds to the dCas9 target site and generates a 3′ overhang double strand break within the dCas9 target site to induce homology-directed repair between the exogenous donor template sequences and the sequences surrounding the break, thereby copying, incorporating, and/or inserting the nucleic acid sequence from the exogenous donor template into the dCas9 target site of the genomic locus of the cell.

In one aspect, the present disclosure relates to a method of copying, incorporating, and/or inserting a nucleic acid sequence from an exogenous donor template into a TALE target site of a genomic locus of a cell, the method including providing an exogenous donor template and a TALE to the nucleus of a cell, where the exogenous donor template includes sequences homologous to sequences within the TALE target site of the genomic locus, and where the TALE nuclease fusion protein binds to the TALE target site and generates a 3′ overhang double strand break within the TALE target site to induce homology-directed repair between the exogenous donor template sequences and the sequences surrounding the break, thereby copying, incorporating, and/or inserting the nucleic acid sequence from the exogenous donor template into the TALE target site of the genomic locus of the cell.

In one aspect, the present disclosure relates to a method of introducing a variable-length insertion or deletion mutation that overlaps with a nuclease target site of a genomic locus of a cell, the method including providing the nucleic acid sequence encoding any of the zinc finger nuclease fusion proteins described herein to the nucleus of a cell, where the zinc finger nuclease fusion protein binds to the nuclease target site and generates a 3′ overhang double strand break within the nuclease target site to induce repair of the break by non-homologous end-joining or microhomology-mediated end joining, thereby leading to the generation of the variable-length insertion or deletion mutation that overlaps with the nuclease target site of the genomic locus of the cell.

In one aspect, the present disclosure relates to a method of introducing a variable-length insertion or deletion mutation that overlaps with a TALE target site of a genomic locus of a cell, the method including providing the nucleic acid sequence encoding any of the TALE nuclease fusion proteins described herein to the nucleus of a cell, where the TALE nuclease fusion protein binds to the TALE target site and generates a 3′ overhang double strand break within the TALE target site to induce repair of the break by non-homologous end-joining or microhomology-mediated end joining, thereby leading to the generation of the variable-length insertion or deletion mutation that overlaps with the TALE target site of the genomic locus of the cell.

In one aspect, the present disclosure relates to a method of introducing a variable-length insertion or deletion mutation that overlaps with a nuclease target site of a genomic locus of a cell, the method including: (a) providing any of the zinc finger nuclease fusion proteins described herein to the nucleus of a cell, where the zinc finger nuclease fusion protein binds to the nuclease target site and (b) generates a 3′ overhang double strand break within the nuclease target site to induce repair of the break by non-homologous end-joining or microhomology-mediated end joining, thereby leading to the generation of the variable-length insertion or deletion mutation that overlaps the nuclease target site of the genomic locus of the cell.

Other features and advantages of the invention will be apparent from the following detailed description and figures, and from the claims.

### DESCRIPTION OF DRAWINGS

The patent or application file contains at least one drawing executed in color. Copies of this patent or patent application publication with color drawing(s) will be provided by the Office upon request and payment of the necessary fee.

FIG. 1 depicts how targeted double-strand breaks (DSBs) induced by genome-editing nucleases led to the formation of variable-length insertion or deletions (indels) by non-homologous end-joining repair or, in the presence of a homologous donor template, of precise sequence modifications or insertions by homology-directed repair (HDR). In most cells, including mammalian cells, nuclease-induced DSBs generally induced indels via NHEJ more efficiently than precise alterations by HDR.

FIG. 2 depicts how dimerization-dependent nuclease domains were fused to catalytically inactive Cas9 (“dead” Cas9 or dCas9) or engineered zinc finger arrays to create dCas9 nucleases or zinc finger nucleases, respectively. When a dimerization-dependent nuclease domain lacking its own DNA-binding specificity was used, the DNA sequence specificities of these fusions were determined by dCas9 complexed with pairs of guide RNAs (gRNAs) or by pairs of DNA binding zinc finger arrays. In the example shown, the nuclease domain was derived from a type IIS restriction enzyme that generated 3′ overhangs at the cleavage sites.

FIGS. 3A-E depict amino acid sequences and identified domains of five type IIS restriction enzymes that generated 3′ overhangs. Type IIS enzymes comprised a nuclease domain and DNA binding domain that were separated by a methyltransferase domain. For all five of the restriction enzymes shown, no precise nuclease domain had been defined and for these cases putative domains indicated based on predictions for the known methyltransferase domain, DNA binding domain, and typical size of nuclease domains for this class of proteins. Putative nuclease domains are underlined, methyltransferase domains are italicized, and DNA binding domains, where defined, are bolded.

FIG. 4 depicts a diagram of the U2OS Traffic Light Reporter (hereafter U2OS.TLR) cell line used to assay DNA repair outcomes induced by targeted nucleases. U2OS.TLR harbored a single integrated copy of the reporter construct illustrated in which a defective copy of EGFP harboring an inactivating point mutation (EGFP*) was expressed from a constitutive EF1alpha (EF1a) promoter. In addition, a T2A-TagRFP fusion was encoded on the same transcript downstream and 2 nucleotides (nts) out of frame (with respect to translation) from the EGFP* gene. Cleavage of a target site within EGFP* and near the inactivating mutation and the resulting introduction of indels via NHEJ led to restoration of the translational reading frame for the T2A-TagRFP gene (note that this is expected to happen with ˜⅓ of the cleavage events assuming that the number of nucleotides introduced or deleted by indels is random).

FIG. 5 depicts how gRNAs was designed in pairs to orient two dCas9 molecules (kidney bean shapes) in either a PAM-Out or PAM-In orientation. Also, note how the length of the “spacer” sequence between the sites bound by the two dCas9 molecules was varied.

FIGS. 6A-J depict the testing of AcuI, AloI, BpmI, BaeI, and MmeI nuclease domains fused to either the amino-terminal or carboxy-terminal end of dSpCas9 using a Gly-Gly-Gly-Gly-Ser (GGGGS (SEQ ID NO: 3)) linker in human cells using U2OS.TLR cells to assay for gene editing activities. These fusions were tested in both PAM-In and PAM-Out orientations with various spacings between binding sites for pairs of guide RNAs complexed with dCas9 fusions. The following fusions were tested in these experiments (with the order of the protein components listed N-terminal to C-terminal): A) dCas9-AcuI; B) AloI-dCas9; C) dCas9-AloI; D) BpmI-dCas9; E) dCas9-BpmI; F) BaeI-dCas9; G) dCas9-BaeI; H) AcuI-dCas9; and I) MmeI-dCas9; J) dCAS9-MmeI. For all experiments shown, FokI-dCas9 with a pair of gRNAs designed to orient the nuclease fusions in a PAM-Out orientation with a 16 bp spacing served as a positive control for gene editing activity. Among all of the fusions and orientations/spacings tested, only the AcuI-dCas9 fusion showed optimal cleavage activity at 17 and 18 bp spacings in the PAM-Out orientation with little activity at any other spacing or orientation (FIG. 6H). AcuI-dCas9 appeared to have a more restricted window of gRNA spacings in which it was active compared to previously published studies using FokI-dCas9 fusions (Tsai et al., Nat Biotech 2014 PMID: 24770325).

FIG. 7 depicts the dependence of AcuI-dCas9 fusion activity on two gRNAs. On-target gRNAs targeted to sites in the EGFP* part of the U2OS.TLR reporter were indicated with (+) symbol while control off-target gRNAs (that did not recognize a sequence in EGFP*) were indicated with (−) symbol. When both on-target gRNAs were present, RFP+ cells were observed for both AcuI-dCas9 and FokI-dCas9 fusions using the U2OS.TLR assay. When one or the other on-target gRNA was replaced with an off-target gRNA, AcuI-dCas9 was no longer recruited to the EGFP* target site as a dimer and cleavage is lost. A similar result was observed with the FokI-dCas9 fusion. Values are average of three independent experiments.

FIG. 8 depicts the activities of AcuI-dCas9 fusions with or without an additional nuclear localization signal (NLS) in the U2OS.TLR assay. Fusions were tested on 16, 17, and 18 bp PAM-Out spacings. FokI-dCas9 on a PAM-Out 16bp spacing was used as a positive control for the assay.

FIG. 9 depicts the activities of AcuI-dCas9 and FokI-dCas9 (both with GGGGS linkers (SEQ ID NO: 3)) at three different human endogenous gene target sites as judged by T7EI assay. The same pairs of gRNAs were used for each target site with AcuI-dCas9 and FokI-dCas9. Results shown were the mean of triplicate samples with error bars reflecting standard error of the mean.

FIG. 10 depicts activities of a truncated AcuI-dCas9 fusion (bearing a shortened AcuI nuclease domain containing only amino acid positions 26-199) in the U2OS.TLR assay. This truncated fusion was tested using pairs of gRNAs with spacings between 0-30 bps in both the PAM-In and PAM-Out orientation. FokI-dCas9 fusion was used as a positive control in this assay and dCas9 alone (not fused to any functional domain) was used as a negative control.

FIG. 11 depicts the genome editing activities of various truncation mutants of the AcuI-dCas9 fusion protein. A series of truncation mutants in which variable numbers of amino acids (AAs) were deleted from the amino-terminal end of the AcuI nuclease domain present in the AcuI-dCas9 fusion (with a GGGGS (SEQ ID NO: 3) linker between the nuclease and the dCas9 domains) were constructed and then compared with “full-length” AcuI-dCas9 and FokI-dCas9 using a pair of gRNAs that target a site (with a spacer of 17 bps between the half-sites) in an integrated constitutively expressed EGFP reporter gene in U2OS cells (U2OS.EGFP cells). Induction of indels by NHEJ-mediated repair of nuclease-induced DNA breaks was expected to result in EGFP-negative cells. Cells expressing the indicated nuclease fusion and the pair of EGFP-targeted gRNAs were assayed for efficiency of EGFP disruption by using flow cytometry. dCas9 with no nuclease domain fused served as a negative control.

FIG. 12 depicts the activities of AcuI-dCas9 fusions bearing XTEN linkers, with and without an NLS, using the U2OS.TLR assay. These fusions were tested with pairs of gRNAs that target PAM-Out sites with spacers ranging from 0 to 31. Note that both fusions showed activities within two spacer ranges of 17-20 bp and 26-29 bps and that the addition of an NLS to the N-terminal end of the AcuI nuclease domain had minimal impact on cleavage activities. Positive and negative controls were the same as in FIG. 10.

FIGS. 13A-B show that AcuI-dCas9 fusions were more efficient for inducing HDR than matched FokI-dCas9 fusions at an integrated reporter gene in human cells. In the experiments of this figure, U2OS.TLR cells were transfected with not only gRNA and dCas9 nuclease fusion (either AcuI-dCas9 or FokI-dCas9) expression vectors but also a single-stranded oligodeoxynucleotide (ssODN) “donor” template that was designed to introduce a restriction enzyme site (BamHI) that can be quantified by a restriction fragment length polymorphism (RFLP) assay. Under these experimental conditions, a nuclease-induced DNA break was able to promote either HDR-mediated introduction of a BamHI restriction site into the EGFP* gene using the ssODN donor template or NHEJ-mediated indel mutations, some of which will result in restoration of TagRFP expression and therefore RFP-positive cells. A) Absolute rates of NHEJ-mediated indels (as judged by percentage RFP-positive cells) and HDR-mediated introduction of a BamHI restriction site (as judged by RFLP) induced by AcuI-dCas9 and FokI-dCas9 using the same pair of GFP-targeted gRNAs (with a 17 bp spacing between the target sites) in human U2OS.TLR cells. Results shown are the mean of duplicate experiments with error bars showing standard errors of the mean. B) Ratios of HDR:NHEJ as measured by RFLP and RFP-positive cells in U2OS.TLR cells for AcuI-dCas9 and FokI-dCas9 using the data from A).

FIGS. 14A-C show that AcuI-dCas9 fusions were more efficient for inducing HDR than matched FokI-dCas9 fusions at various endogenous gene target sites in human cells. Vectors encoding pairs of gRNAs that target sites with 17 or 18 bp spacers in the endogenous human FANCF, BRCA1, DDB2, and EMX1 genes were introduced into U2OS human cells together with another vector expressing either AcuI-dCas9 or FokI-dCas9 and with or without a ssODN donor template designed to insert a BamHI restriction site at the site of cleavage. (A) Absolute rates of HDR-mediated introduction of a BamHI restriction site (as judged by RFLP). (B) NHEJ-mediated indels (as judged T7 Endonuclease I (T7EI) assays) induced by AcuI-dCas9 and FokI-dCas9 using the same pair of gRNAs designed for each of the four different endogenous gene target sites with or without a ssODN donor template. (C) Fold-change in the ratios of HDR:NHEJ as measured by RFLP and T7EI assays in (A) and (B) for AcuI-dCas9 and FokI-dCas9 in the presence of gRNA pairs and a cognate ssODN donor template.

FIG. 15 depicts fusions of engineered zinc finger arrays to the FokI or AcuI nuclease domains. In the examples shown, the nuclease domains were fused to the carboxy-terminal end of the engineered zinc finger arrays; however, it was also possible that nuclease domains could have been fused on the amino-terminal end of the engineered zinc finger arrays as well.

FIG. 16 depicts a bacterial screening method for assaying the activities of engineered zinc finger array-AcuI fusions (hereafter ZF-AcuI fusions). A ccdB-sensitive E. coli strain was transformed with the toxic plasmid (which contained a toxic ccdB gene expressed from an arabinose-inducible promoter (pBAD) and binding sites for engineered zinc finger arrays positioned downstream of the ccdB gene). Expression of a zinc finger array (fused to the AcuI nuclease domain or FokI nuclease domain) that can recognize and cleave a palindromic version of its target site in this strain would have led to cleavage of the plasmid encoding the toxic ccdB gene, resulting in its degradation and thereby permitting cell survival under conditions in which ccdB gene expression was induced. Colony survival on selective media was therefore a measure of cleavage of the toxic plasmid by the zinc finger array-AcuI nuclease domain fusion. Cleavage was measured as % colony survival between Arabinose containing media, where ccdB was expressed, and media lacking arabinose, where ccdB was not expressed.

FIG. 17 depicts the cleavage activities of zinc finger-AcuI fusions harboring an LRGS linker on palindromic target sites with a 7 bp spacing between those sites in the bacterial assays illustrated in FIG. 16 above. Data for four different zinc finger arrays (each consisting of three fingers engineered to work together to recognize a 9-10 bp target site) fused to either FokI or AcuI nuclease domains are shown. Survival was calculated based on colony count on selective media (with Arabinose) divided by colony count on non-selective (without Arabinose) media.

FIG. 18 depicts the activities of various engineered zinc finger arrays fused to either AcuI or FokI nuclease domain on target sites with 6 bp spacers between palindromic binding sites for the zinc finger arrays in the bacterial cell-based assay described above in FIG. 16. Percentage survival was calculated as described in FIG. 17 above.

FIG. 19 depicts the gene editing activities in human cells of zinc finger array-AcuI nuclease domain fusions linked by either LRGS linker or directly with no linker on target sites with 6 bp spacers between target “half-sites”. Pairs of zinc finger arrays previously designed to target half-sites with 6 bp spacer sequences in the EGFP gene (Maeder et al., Mol Cell 2008, PMID: 18657511) were used to construct the AcuI nuclease fusions. The capabilities of these pairs of zinc finger array-AcuI nuclease domain fusions to induce gene editing events were assessed using the human U2OS cell-based EGFP disruption assay described in FIG. 11 above. For positive controls, these same pairs of engineered zinc finger arrays fused to the FokI nuclease domain by an LRGS were tested. These fusions were previously shown to be efficient for cleaving the EGFP gene (Maeder et al., Mol Cell 2008, PMID: 18657511). U2OS.EGFP cells transfected with an empty ZF-nuclease fusion expression plasmid served as the negative control. (Note that in all of the FokI and AcuI fusions tested, the nuclease domain was fused to the carboxy-terminal end of the zinc finger array.)

FIG. 20 shows assessment of cleavage at target site for MmeI-dCas9 fusion protein (MmeI endonuclease domain fused to N or C terminal end of dCas9) with 16, 17, and 23 bps gRNAs using T7E1 assay.

FIG. 21 depicts the fusion of AcuI to the N or C terminal end of Transcription activator-like effectors (TALEs). Dimerization and recruitment of AcuI to the target site in a sequence-dependent manner is mediated by the sequence specificity of a pair of TALEs.

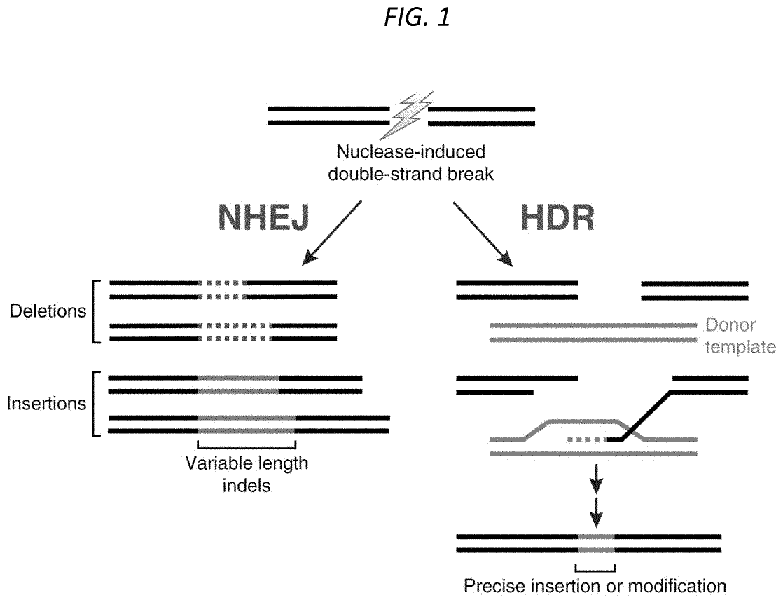

### DETAILED DESCRIPTION

### Definitions

Unless otherwise defined, all technical and scientific terms used herein have the same meaning as commonly understood by one of ordinary skill in the art to which this invention belongs. In case of conflict, the present application, including definitions will control.

As used herein, the term zinc finger refers to refers to a polypeptide comprising a DNA binding domain that is stabilized by zinc. The individual DNA binding domains are typically referred to as “fingers.” A zinc finger protein has at least one finger, preferably two fingers, three fingers, four fingers, five fingers, or six fingers. A zinc finger protein having two or more zinc fingers is referred to as a “multi-finger” or “multi-zinc finger” protein or “multi-finger array” or “zinc finger array.” Each finger typically comprises an approximately 30 amino acid, zinc-chelating, DNA-binding domain. An exemplary motif characterizing one class of these proteins is X(2)-Cys-X(2,4)-Cys-X(12)-His-X(3-5)-His (SEQ ID NO:1), where X is any amino acid, which is known as the “C(2)H(2)” class. Studies have demonstrated that a single zinc finger of this C(2)H(2) class consists of an alpha helix containing the two invariant histidine residues coordinated with zinc along with the two cysteine residues of a single beta turn (Berg and Shi, Science 271:1081-1085 (1996)). Each finger within a zinc finger protein binds to about two to about five base pairs within a DNA sequence.

As used herein, the term “zinc finger fusion protein” refers to at least one zinc finger fused (i.e., joined), optionally through an amino acid linker, to a functional domain. A zinc finger 3′-overhang nuclease fusion protein comprises a zinc finger fused to nuclease domain, where the nuclease domain generates 3′ overhang double strand breaks (i.e., a cleavage site in a double stranded DNA which leaves a 3′ overhanging end).

As used herein, a “dimerization-dependent nuclease domain” is a domain having DNA nuclease activity upon dimerization (a dimer is a complex formed by two, usually non-covalently bound, monomer proteins). The nuclease activity can be, for example, that which that generates 3′ overhang double strand breaks in DNA.

As used herein, a “C-terminal zinc finger nuclease” refers to a nuclease domain located in the C-terminal or carboxy-terminal portion of a protein or zinc finger fusion protein.

A “target site” or “target sequence” is a nucleic acid sequence that defines a portion of a nucleic acid to which a binding molecule will bind, provided sufficient conditions for binding exist. As used herein, a “target site” or “nuclease target site” of a genomic locus comprises: i) sequences homologous to an exogenous “donor template” nucleic acid sequence, which is to be copied, inserted and/or incorporated within the target site, ii) sequences to which zinc fingers bind, and iii) sequences cleaved by nucleases that generate 3′ overhang double strand breaks. A nucleic acid sequence that is “copied” refers to duplication of that sequence within the target site; a nucleic acid sequence that is “inserted” refers to adding that sequence within the target site; and a nucleic acid sequence that is “incorporated” refers to replacement of a nucleic acid sequence within the target site with the incorporated sequence.

An “exogenous” nucleic acid sequence is a nucleic acid sequence that is not normally present in a cell, but can be introduced into a cell by one or more genetic, biochemical or other methods. Normal presence in the cell is determined with respect to the particular developmental stage and environmental conditions of the cell. Thus, for example, as used herein, an extrachromosomal DNA sequence that is introduced into the cell is an exogenous nucleic acid (even if part or all of that sequence is also present in the genome of the cell). Similarly, a nucleic acid sequence that is present only during embryonic development of muscle is an exogenous nucleic acid sequence with respect to an adult muscle cell. Alternatively, a nucleic acid sequence induced by heat shock is an exogenous molecule with respect to a non-heat-shocked cell. An exogenous nucleic acid sequence can comprise, for example, a functioning version of a malfunctioning endogenous gene. By contrast, an “endogenous” nucleic acid sequence is one that is normally present in a particular cell at a particular developmental stage under particular environmental conditions. For example, an endogenous nucleic acid can comprise a chromosome, the genome of a mitochondrion, chloroplast or other organelle, or a naturally-occurring episomal nucleic acid.

The term “donor template” refers to an exogenous double-stranded or single-stranded nucleic acid sequence that is used to be copied, incorporated, and/or inserted during the repair of double-strand breaks comprising for example, a sequence alteration of interest to create one or more base changes in a target site or a sequence resulting in a more lengthy insertion or deletion at or near a nuclease target site.

“Nucleic acid” refers to deoxyribonucleotides or ribonucleotides in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs). Unless otherwise indicated, a particular nucleic acid sequence also implicitly encompasses conservatively modified variants thereof (e.g., degenerate codon substitutions) and complementary sequences, as well as the sequence explicitly indicated. The term nucleic acid is used interchangeably with gene, cDNA, mRNA, oligonucleotide, and polynucleotide. A “gene,” for the purposes of the present disclosure, includes a DNA region encoding a gene product (see infra), as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites and locus control regions.

The terms “polypeptide,” “peptide” and “protein” are used interchangeably herein to refer to a polymer of amino acid residues. The terms apply to amino acid polymers in which one or more amino acid residue is an analog or mimetic of a corresponding naturally occurring amino acid, as well as to naturally occurring amino acid polymers.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analog refers to compounds that have the same basic chemical structure as a naturally occurring amino acid, i.e., an α-carbon that is bound to a hydrogen, a carboxyl group, an amino group, and an R group, e.g., homoserine, norleucine, methionine sulfoxide, methionine, and methyl sulfonium. Such analogs have modified R groups (e.g., norleucine) or modified peptide backbones, but retain the same basic chemical structure as a naturally occurring amino acid.

Homology-directed repair is a mechanism in cells to repair double strand DNA breaks via homologous recombination (HR), single-stranded annealing (SSA), or other mechanisms in which a homologous template is used in the repair. As used herein, the term “homology-directed repair (HDR)” refers to DNA repair that takes place in cells, for example, during repair of double-strand breaks in DNA. HDR requires nucleotide sequence homology and uses a donor template, such as an exogenous donor nucleic acid sequence (that can be either single-stranded or double-stranded), to repair the sequence where the double-strand break occurred (e.g., target site or sequence). This results in the transfer of genetic information from, for example, the donor template to the target sequence. HDR may result in alteration of the target sequence (e.g., insertion, deletion, mutation, correction) if the donor template sequence differs from the target sequence and part or all of the sequence information from the donor template is incorporated or copied into the target sequence.

As used herein, the term “non-homologous end-joining” refers to repairs made to double-strand breaks in DNA, whereby the break ends are directly ligated without the need for a homologous template, in contrast to homology directed repair. NHEJ typically utilizes endogenous nucleic acid sequences to guide repair (e.g., single-stranded overhangs on the ends of double-strand breaks). Imprecise repair leading to loss of nucleotides can occur when the overhangs are not compatible, creating insertions and deletions.

As used herein, the term “microhomology-mediated end joining” refers to the annealing of homologous or partially homologous endogenous nucleic acid sequences (e.g., about 5-25 base pair sequences) during the alignment of processed overhangs that are generated after a 3′ double strand break and before re-joining, thereby resulting in insertions and deletions flanking the original break.

A “Type IIS restriction enzyme”, as used here in, is a restriction enzyme that recognizes asymmetric DNA sequences and cleaves outside of their recognition sequence. In one embodiment, the restriction enzyme is AcuI.

As used herein, the terms “treat,” “treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

The term “Cas protein” as used herein refers to Type II CRISPR-Cas proteins, including, but not limited to Cas9, Cas9-like, Cas1, Cas2, Cas3, Csn2, Cas4, proteins encoded by Cas9 orthologs, Cas9-like synthetic proteins, and variants and modifications thereof. The term “Cas9 protein” as used herein refers to Cas9 wild-type proteins derived from Type II CRISPR-Cas9 systems, modifications of Cas9 proteins, variants of Cas9 proteins, Cas9 orthologs, and combinations thereof. As used herein, a “catalytically inactive Cas9 domain” refers to a polypeptide domain of Cas9 that is lacking endonuclease activity, for example, by introducing point mutations in catalytic residues (D10A and H840A) of the gene encoding Cas9. In doing so, the “dCas9,” or dead Cas9, domain is unable to cleave dsDNA but retains the ability to associate with a guide RNA (or complex of crRNA and tracrRNA) and to target DNA.

The term “Cas9 target site” or “dCas9 target site” refer to a genomic locus that comprises a sequence that is complementary to the dCas9 guide RNA (which is comprised of a tracrRNA and crRNA) with an adjoining protospacer adjacent motif (PAM) sequence recognized by the Cas9 or dCas9 protein.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (as well as fractions thereof unless the context clearly dictates otherwise).

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

Other definitions appear in context throughout this disclosure.

### Compositions and Methods

Described herein are DNA-binding domain (DBD) nuclease fusion proteins and methods of using the same for enhancing homology-directed repair frequencies at the site of a nuclease-induced double strand breaks for use in genome editing.

The DBD is a protein or a protein domain that binds to its target nucleic acid in a sequence-dependent manner. Described herein are DBD nuclease fusion protein where the DBD is either a zinc finger array or a dCas9.

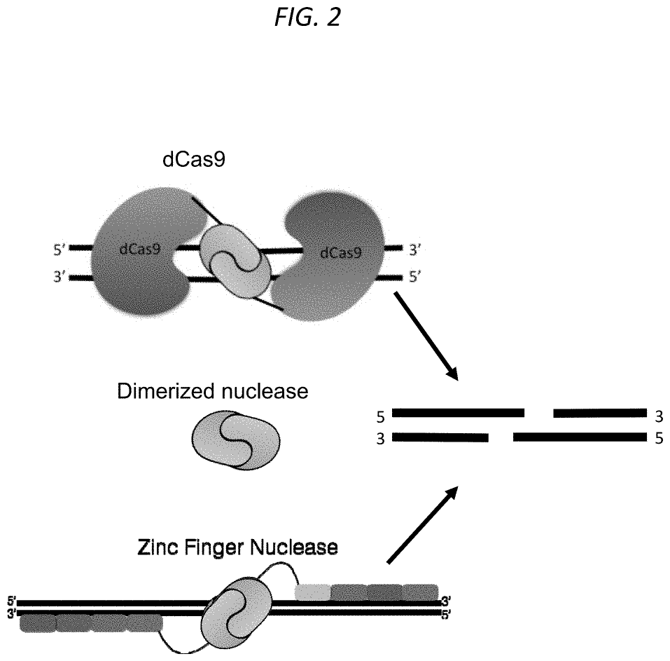

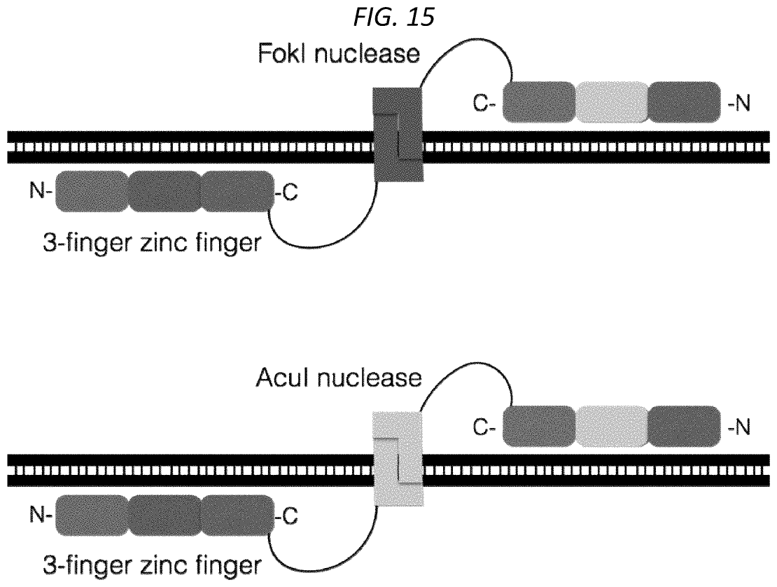

The zinc finger nuclease fusion proteins described herein comprise a nuclease domain that generates a 3′ overhang double strand break in DNA upon dimerization (i.e., the nuclease activity is “dimerization-dependent”); an optional amino acid linker; and a zinc finger domain comprising one or more carboxy-terminal or amino-terminal zinc finger(s). Zinc finger nuclease fusion proteins in the monomer form, comprising one or more carboxy-terminal or amino-terminal zinc finger(s), join together to form a dimer either upon or prior to binding to a target site (FIG. 2; FIG. 15), thereby activating the nuclease cleavage.

The zinc finger nuclease fusion proteins described herein can be used to create insertion/deletion mutations (indels) with high frequency via repair of nuclease-induced DNA breaks by non-homologous end-joining. Zinc finger nuclease fusion proteins can also be used to copy, incorporate, or insert an exogenous nucleic acid sequence of interest into a target site of a genomic locus of a cell. In some embodiments, these methods comprise providing to the nucleus of a cell an exogenous nucleic acid “donor template” sequence and another nucleic acid sequence encoding the zinc finger nuclease fusion protein or the fusion protein itself. The exogenous nucleic acid donor template sequence comprises end sequences homologous to sequences within the target site of the genomic locus. Zinc fingers are designed to recognize and bind to the genomic target site with specificity. Upon binding to the target site, the dimerized nuclease domains of the fusion protein(s) generates a 3′ overhang double strand break within the target site to induce homology-directed repair between sequences surrounding the break and the exogenous nucleic acid sequence, thereby copying, incorporating and/or inserting the exogenous nucleic acid sequence into the target site of the genomic locus of the cell.

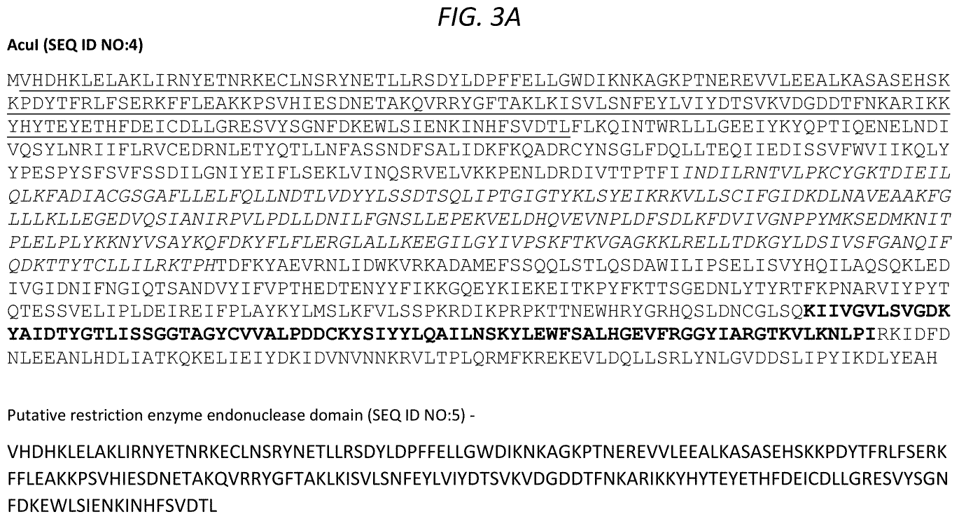

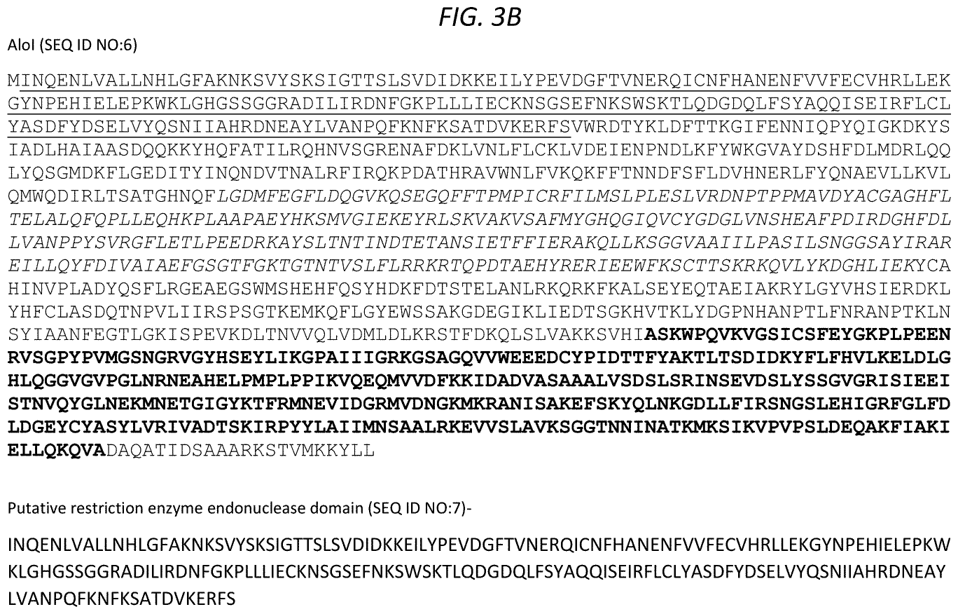

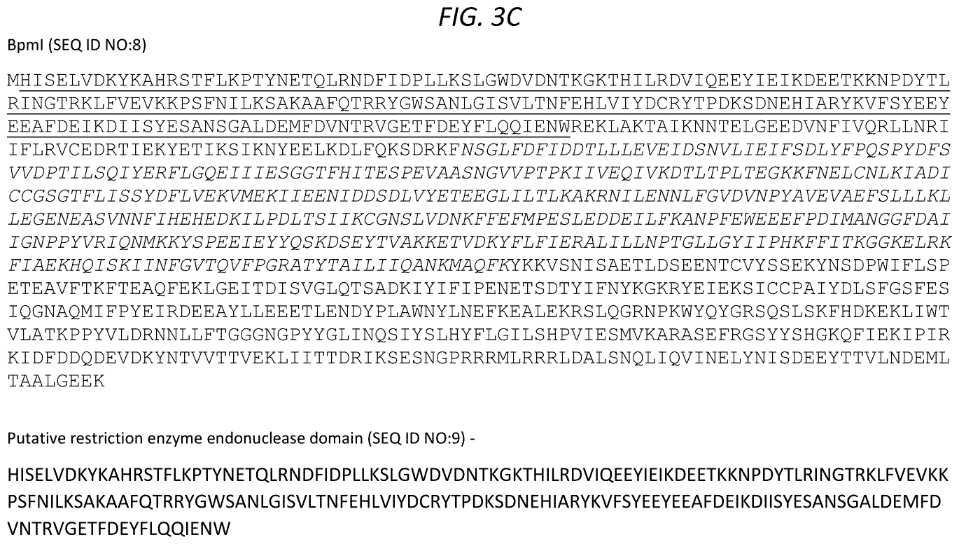

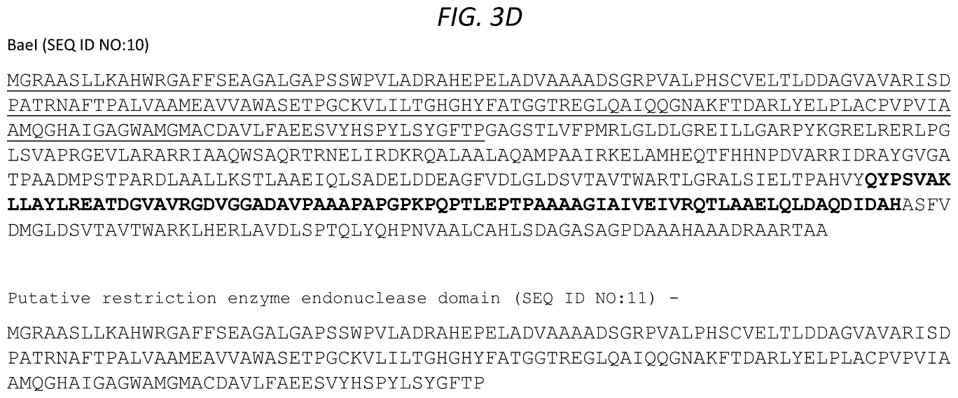

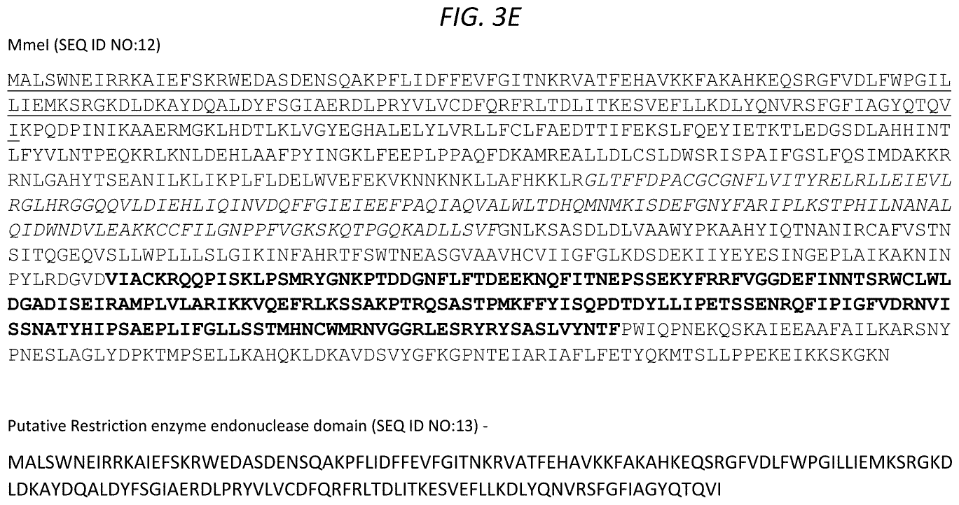

Zinc finger nuclease fusion proteins can comprise any nuclease domain capable of generating a 3′ overhang double strand break in DNA upon dimerization. The nuclease domain can be, for example, a Type IIS restriction enzyme nuclease domain including, but not limited to a AcuI, AloI, BpmI, BaeI, or MmeI nuclease domain. In some instances, the AcuI nuclease domain can have an amino acid sequence. Exemplary amino acid sequences of AcuI, AloI, BpmI, BaeI, or MmeI are shown in FIGS. 3A, 3B, 3C, 3D, and 3E, respectively).

Exemplary nucleotide and amino acid sequences encoding AcuI are known in the art and can be located, for example, at GenBank accession number HQ327692.1.

In some embodiments, the Type IIS restriction enzyme nuclease domain includes isoschizomers of AcuI, e.g., Eco57I. The nucleotide and amino acid sequences encoding Eco57I can be located, for example at UniProt database reference number P25239.

Exemplary nucleotide and amino acid sequences encoding AloI are known in the art and can be located, for example, at GenBank accession number AJ312389.1.

Exemplary nucleotide and amino acid sequences encoding BpmI are known in the art and can be located, for example, at GenBank accession number ADK30556.1.

Exemplary nucleotide and amino acid sequences encoding BaeI are known in the art and can be located, for example, at GenBank accession number ABS74060.1.

Exemplary nucleotide and amino acid sequences encoding MmeI are known in the art and can be located, for example, at GenBank accession number EU616582.1.

Any Type IIS restriction enzyme nuclease domain having dimerization-dependent nuclease activity could be fused to a zinc finger domain and used to conduct the methods described herein. In some embodiments, the nuclease domain is attached to the C-terminus of the zinc finger domain. In other embodiments, the nuclease domain is attached to the N-terminus of the zinc finger domain.

Zinc finger nuclease fusion proteins can further comprise any zinc finger domain constructed according to methods known in the art. Zinc fingers are engineered to recognize a selected target site within a genomic locus. Any suitable method known in the art can be used to design and construct nucleic acids encoding zinc fingers, e.g., phage display, random mutagenesis, combinatorial libraries, computer/rational design, affinity selection, PCR, cloning from cDNA or genomic libraries, synthetic construction and the like. The following US patent publications comprehensively describe methods for design, construction, and expression of zinc fingers for selected target sites and are incorporated herein by reference: U.S. Ser. Nos. 70/13,219, 67/46,838, 72/41,573, 68/66,997, 67/85,613, 72/41,574, 67/94,136, 70/30,215, 64/53,242, 65/34,261, US Patent Publication No. 20120178647, US Patent Publication No. 20070178454, US Patent Publication No. 20060246440, U.S. Ser. Nos. 61/40,081, 62/42,568, 66/10,512, 71/01,972, 73/29,541, 61/40,466, 67/90,941, 57/89,538, and 63/65,379.

The zinc finger domain can also be derived from zinc fingers known in the art and engineered to bind to target sequences within a genomic locus associated with a heritable disease or the progression of a disease, such as cancer. Such zinc fingers have been described, for example, by Umov F D, et al. Nat Rev Genet. 2010 September; 11(9):636-46; Chang K H, et al. Mol Ther Methods Clin Dev. 2017 Jan. 11; 4:137-148; Beane J D, et al. Mol Ther. 2015 August; 23(8):1380-90 and Tebas P, N Engl J Med. 2014 Mar. 6; 370(10):901-10.

The dimerization-dependent nuclease domain and the zinc finger domain of the zinc finger nuclease fusion protein can be joined together by an amino acid linker. The terms linked, joined and fused are used interchangeably herein to refer to the means by which two domains of a fusion protein are joined. The amino acid linker can comprise any sequence of at least one amino acid and up to a sequence of 10 amino acids. In specific embodiments, the linker can comprise Leucine, Arginine, Glycine and Serine (LRGS (SEQ ID NO:2)); glycine, glycine, glycine, glycine and serine (GGGGS (SEQ ID NO:3)); or a non-standard amino acid, threonine, glutamic acid and asparagine (XTEN) as described by Shellenberger, et al. Nat Biotechnol. 2009 December; 27(12):1186-90.

In some embodiments, the dimerization-dependent nuclease domain, the zinc finger domain, the TALE, and/or the dCas9 domain can have an amino acid sequences that have at least 80%, at least 85%, at least 90%, at least 95%, at least 96%, at least 97%, at least 98%, or at least 99% sequence identity to the amino acid sequence of the exemplary amino acid sequences of the dimerization-dependent nuclease domain, the zinc finger domain, the TALE, and/or the dCas9, described herein.

In some embodiments, the dimerization-dependent nuclease domain, the zinc finger domain, the TALE, and/or the dCas9 domain can be encoded by a nucleic acid sequences that have at least 80%, at least 85%, at least 90%, at least 95%, least 96%, at least 97%, at least 98%, or at least 99% sequence identity to the exemplary nucleic acid sequences encoding the dimerization-dependent nuclease domain, the zinc finger domain, the TALE, and/or the dCas9, described herein.

To determine the percent identity of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The length of a reference sequence aligned for comparison purposes is at least 80% of the length of the reference sequence, and in some embodiments is at least 90% or 100%. The nucleotides at corresponding amino acid positions or nucleotide positions are then compared. When a position in the first sequence is occupied by the same nucleotide as the corresponding position in the second sequence, then the molecules are identical at that position (as used herein nucleic acid “identity” is equivalent to nucleic acid “homology”). The percent identity between the two sequences is a function of the number of identical positions shared by the sequences, taking into account the number of gaps, and the length of each gap, which need to be introduced for optimal alignment of the two sequences. Percent identity between two polypeptides or nucleic acid sequences is determined in various ways that are within the skill in the art, for instance, using publicly available computer software such as Smith Waterman Alignment (Smith, T. F. and M. S. Waterman (1981) J Mol Biol 147:195-7); “BestFit” (Smith and Waterman, Advances in Applied Mathematics, 482-489 (1981)) as incorporated into GeneMatcher Plus™, Schwarz and Dayhof (1979) Atlas of Protein Sequence and Structure, Dayhof, M. O., Ed, pp 353-358; BLAST program (Basic Local Alignment Search Tool; (Altschul, S. F., W. Gish, et al. (1990) J Mol Biol 215: 403-10), BLAST-2, BLAST-P, BLAST-N, BLAST-X, WU-BLAST-2, ALIGN, ALIGN-2, CLUSTAL, or Megalign (DNASTAR) software. In addition, those skilled in the art can determine appropriate parameters for measuring alignment, including any algorithms needed to achieve maximal alignment over the length of the sequences being compared. In general, for proteins or nucleic acids, the length of comparison can be any length, up to and including full length (e.g., 5%, 10%, 20%, 30%, 40%, 50%, 60%, 70%, 80%, 90%, 95%, or 100%). For purposes of the present compositions and methods, at least 80% of the full length of the sequence is aligned.

For purposes of the present invention, the comparison of sequences and determination of percent identity between two sequences can be accomplished using a Blossum 62 scoring matrix with a gap penalty of 12, a gap extend penalty of 4, and a frameshift gap penalty of 5.

Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine.

Upon binding to the target site and forming a dimer complex, the nuclease domain of the zinc finger nuclease fusion protein generates a 3′ overhang double strand break within the target site to induce homology-directed repair, with resulting copying, incorporating, and/or integrating of the exogenous nucleic acid sequence, or a portion thereof, within the target site. Where there is nucleotide sequence homology, a donor template oligonucleotide sequence (either single- or double-stranded) can act as a template to repair a target DNA sequence that experienced the double-strand break, leading to the transfer of genetic information from the donor to the target. Such transfer can involve mismatch correction of heteroduplex DNA that forms between the broken target and the donor, and/or synthesis-dependent strand annealing, in which the donor is used to re-synthesize genetic information that will become part of the target, and/or related processes. Homology-directed repair often results in an alteration of the sequence of the target nucleotide such that part or all of the sequence of the donor nucleotide sequence is copied and/or incorporated into the target nucleotide.

The zinc finger nuclease fusion protein creates a double-stranded break in the target sequence at a predetermined site, and an exogenous nucleic acid sequence acting as a donor template, having homology to the nucleotide sequence in the region of the break, can be copied, incorporated, and/or introduced into the genomic locus. The presence of the double-stranded break has been shown to greatly enhance the efficiencies of these different repair outcomes. The donor sequence may be physically integrated or, alternatively, the donor nucleotide is used as a template for repair of the break via homologous recombination, resulting in the introduction of all or part of the nucleotide sequence as in the donor into the genomic locus. Thus, a sequence in the genomic locus can be altered and, in certain embodiments, can be converted into a sequence present in a donor nucleotide.

Also described herein are dCas9 nuclease fusion proteins and methods of using the same for enhancing homology-directed repair frequencies at the site of a nuclease-induced double strand breaks. dCas9 nuclease fusion proteins comprise a catalytically inactive Cas9 carboxy-terminal or amino-terminal domain linked to a dimerization-dependent nuclease domain that generates 3′ overhang double strand breaks in DNA. A catalytically inactive Cas9 domain contains mutations (e.g., D10A and/or H841A) which results in the loss of native endonuclease activity (Qi et al., Cell (2013)). The endonuclease activity is instead provided by the linked dimerization-dependent nuclease domain to which it is fused. dCas9 nuclease fusion proteins in the monomer form join together to form a dimer either prior to or upon binding to a dCas9 target site, thereby activating the nuclease cleavage.

Clustered regularly interspaced short palindromic repeats (CRISPR) and associated Cas proteins constitute the CRISPR-Cas system. The RNA-guided Cas9 endonuclease specifically targets and cleaves DNA in a sequence-dependent manner (Gasiunas, G., et al., Proc Natl Acad Sci USA 109, E2579-E2586 (2012); Jinek, M., et al., Science 337, 816-821 (2012); Sternberg, S. H., et al., Nature 507, 62 (2014); Deltcheva, E., et al., Nature 471, 602-607 (2011)), and has been widely used for programmable genome editing in a variety of organisms and model systems (Cong, L., et al., Science 339, 819-823 (2013); Jiang, W., et al., Nat. Biotechnol. 31, 233-239 (2013); Sander, J. D. & Joung, J. K., Nature Biotechnol. 32, 347-355. (2014)). Cas9 requires a guide RNA composed of two RNAs that associate or are covalently linked together to make a guide RNA; the CRISPR RNA (crRNA), and the trans-activating RNA (tracrRNA). If the nucleotide sequence of a genomic locus of interest is complementary to the guide RNA, Cas9 recognizes and cleaves the site. A ternary complex of Cas9 with crRNA and tracrRNA or a binary complex of Cas9 with a guide RNA can bind to and cleave dsDNA protospacer sequences that match the crRNA spacer and that are also adjoined to a short protospacer-adjacent motif dCas9 can still associate with a crRNA/tracrRNA complex or with a guide RNA and then recognize and bind to a target site even though its native catalytic activity is inactivated. The nucleotide and amino acid sequences encoding Cas9 are known in the art and can be located, for example, at GenBank accession number NC_002737.2.

dCas9 nuclease fusion proteins described herein can be used to induce homology-directed repair events at a target site of a genomic locus of a cell. This method comprises providing an exogenous nucleic acid sequence, a nucleic acid sequence encoding the dCas9 nuclease fusion protein and one or more (e.g., at least two) guide RNAs to the nucleus of a cell. The exogenous nucleic acid sequence comprises end sequences homologous to sequences within the target site of the genomic locus. The guide RNA is designed to direct two dCas9 nuclease fusions to a predetermined target site in which each dCas9/gRNA complex binds to one of two “half-sites”. The dCas9 domains will recognize and bind to their target sites with complementary to the guide RNA and an adjoining PAM sequence with specificity. Upon binding to the target site, the linked nuclease domain of the fusion protein functions as a dimer to generate a 3′ overhang double strand break within the target site to induce homology-directed repair between sequences surrounding the break and the exogenous nucleic acid sequence, thereby copying, incorporating, and/or inserting the exogenous nucleic acid sequence into the target site of the genomic locus of the cell. The nucleotide and amino acid sequences encoding dCas9 are known in the art and can be located, for example, at GenBank accession number KR011748.1. dCas9 is also described by Zetsche et al., Nature Biotechnology 33, 139-142 (2015).

dCas9 nuclease fusion proteins can comprise any nuclease domain capable of generating a 3′ overhang double strand break in DNA upon dimerization. The nuclease domain can be, for example, a Type IIS restriction enzyme nuclease domain including, but not limited to a AcuI, AloI, BpmI, BaeI, or MmeI nuclease domain. The dimerization-dependent nuclease domain and the dCas9 domain of the dCas9 nuclease fusion proteins are joined together by an optional amino acid linker. The amino acid linker can comprise any sequence of at least one amino acid and up to a sequence of 10 amino acids. In specific embodiments, the amino acid linker can comprise, for example glycine, glycine, glycine, glycine and serine (GGGGS (SEQ ID NO:3)) or a non-standard amino acid, threonine, glutamic acid and asparagine (XTEN).

In any of the methods and compositions described herein, the exogenous nucleotide sequence acting as a donor can contain sequences that are homologous, but not identical, to genomic sequences in the target site, thereby stimulating homology-directed repair to copy, incorporate, and/or insert a non-identical sequence within the target site. Thus, in certain embodiments, portions of the donor sequence that are homologous to sequences in the region of interest exhibit between about 80 to 99% (or any integer therebetween) sequence identity to the genomic sequence that is replaced. In other embodiments, the homology between the donor and genomic sequence is higher than 99%, for example if only 1 nucleotide differs as between donor and genomic sequences of over 100 contiguous base pairs. In certain cases, a non-homologous portion of the donor sequence can contain sequences not present in the target site, such that new sequences are introduced into the region of interest. In these instances, the non-homologous sequence is generally flanked by sequences of 50-1,000 base pairs (or any integral value there between) or any number of base pairs greater than 1,000, that are homologous or identical to sequences in the target site.

In some embodiments, an entire donor template sequence or a portion of the donor template sequence is integrated at the target site. Any of the methods described herein can be used for partial or complete inactivation of one or more genomic loci in a cell by targeted integration of donor sequence that disrupts expression of the gene(s) of interest. Any of the methods described herein can be used to replace mutated sequences within the target site, thereby correcting a mutated gene or inducing formerly inactive gene expression. The nature of the exogenous nucleic acid sequence to be incorporated will depend on the therapeutic goal to be achieved and can range from inducing or inhibiting gene transcription, to replacing mutated sequences of a defective gene or adding or deleting sequences within a gene.

In other embodiments, the DBD (e.g., zinc finger or dCas9) nuclease fusion protein introduces a variable-length insertion or deletion mutation that overlaps, partially or completely, with a nuclease target site of a genomic locus of a cell through non-homologous end-joining or microhomology-mediated end joining. In these embodiments, no exogenous donor sequence is provided. Rather, a nucleic acid sequence encoding a zinc finger nuclease fusion protein or an isolated zinc finger nuclease fusion protein is provided to the nucleus of a cell, and the zinc finger nuclease fusion protein binds to the nuclease target site to generate a 3′ overhang double strand break within the nuclease target site, followed by repair of the break by non-homologous end-joining or microhomology-mediated end joining. Both non-homologous end-joining or microhomology-mediated end joining can produce insertions or deletions that interfere with, or inhibit, gene transcription at the nuclease target site.

### Delivery and Expression Systems

To use the DBD nuclease fusion protein described herein, it may be desirable to express them from a nucleic acid that encodes them. This can be performed in a variety of ways. For example, the nucleic acid encoding the DBD (e.g., zinc finger or /dCas9) nuclease fusion protein can be cloned into an intermediate vector for transformation into prokaryotic or eukaryotic cells for replication and/or expression. Intermediate vectors are typically prokaryote vectors, e.g., plasmids, or shuttle vectors, or insect vectors, for storage or manipulation of the nucleic acid encoding the DBD nuclease fusion protein for production of the DBD nuclease fusion protein. The nucleic acid encoding the DBD nuclease fusion protein can also be cloned into an expression vector, for administration to a plant cell, animal cell, preferably a mammalian cell or a human cell, fungal cell, bacterial cell, or protozoan cell.

To obtain expression, a sequence encoding a DBD nuclease fusion protein is typically subcloned into an expression vector that contains a promoter to direct transcription. Suitable bacterial and eukaryotic promoters are well known in the art and described, e.g., in Sambrook et al., Molecular Cloning, A Laboratory Manual (3d ed. 2001); Kriegler, Gene Transfer and Expression: A Laboratory Manual (1990); and Current Protocols in Molecular Biology (Ausubel et al., eds., 2010). Bacterial expression systems for expressing the engineered protein are available in, e.g., E. coli, Bacillus sp., and Salmonella (Palva et al., 1983, Gene 22:229-235). Kits for such expression systems are commercially available. Eukaryotic expression systems for mammalian cells, yeast, and insect cells are well known in the art and are also commercially available.

The promoter used to direct expression of a nucleic acid depends on the particular application. For example, a strong constitutive promoter is typically used for expression and purification of fusion proteins. In contrast, when the DBD nuclease fusion protein is to be administered in vivo for gene regulation, either a constitutive or an inducible promoter can be used, depending on the particular use of the DBD nuclease fusion protein. In addition, a preferred promoter for administration of the DBD nuclease fusion protein can be a weak promoter, such as HSV TK or a promoter having similar activity. The promoter can also include elements that are responsive to transactivation, e.g., hypoxia response elements, Gal4 response elements, lac repressor response element, and small molecule control systems such as tetracycline-regulated systems and the RU-486 system (see, e.g., Gossen & Bujard, 1992, Proc. Natl. Acad. Sci. USA, 89:5547; Oligino et al., 1998, Gene Ther., 5:491-496; Wang et al., 1997, Gene Ther., 4:432-441; Neering et al., 1996, Blood, 88:1147-55; and Rendahl et al., 1998, Nat. Biotechnol., 16:757-761).

In addition to the promoter, the expression vector typically contains a transcription unit or expression cassette that contains all the additional elements required for the expression of the nucleic acid in host cells, either prokaryotic or eukaryotic. A typical expression cassette thus contains a promoter operably linked, e.g., to the nucleic acid sequence encoding the DBD nuclease fusion protein, and any signals required, e.g., for efficient polyadenylation of the transcript, transcriptional termination, ribosome binding sites, or translation termination. Additional elements of the cassette may include, e.g., enhancers, and heterologous spliced intronic signals.

The particular expression vector used to transport the genetic information into the cell is selected with regard to the intended use of the DBD nuclease fusion protein t, e.g., expression in plants, animals, bacteria, fungus, protozoa, etc. Standard bacterial expression vectors include plasmids such as pBR322 based plasmids, pSKF, pET23D, and commercially available tag-fusion expression systems such as GST and LacZ.

Expression vectors containing regulatory elements from eukaryotic viruses are often used in eukaryotic expression vectors, e.g., SV40 vectors, papilloma virus vectors, and vectors derived from Epstein-Barr virus. Other exemplary eukaryotic vectors include pMSG, pAV009/A+, pMTO10/A+, pMAMneo-5, baculovirus pDSVE, and any other vector allowing expression of proteins under the direction of the SV40 early promoter, SV40 late promoter, metallothionein promoter, murine mammary tumor virus promoter, Rous sarcoma virus promoter, polyhedrin promoter, or other promoters shown effective for expression in eukaryotic cells.

The vectors for expressing the DBD nuclease fusion protein can include RNA Pol III promoters to drive expression of the guide RNAs, e.g., the H1, U6 or 7SK promoters. These human promoters allow for expression of DBD nuclease fusion proteins in mammalian cells following plasmid transfection.

Some expression systems have markers for selection of stably transfected cell lines such as thymidine kinase, hygromycin B phosphotransferase, and dihydrofolate reductase. High yield expression systems are also suitable, such as using a baculovirus vector in insect cells, with the gRNA encoding sequence under the direction of the polyhedrin promoter or other strong baculovirus promoters.

The elements that are typically included in expression vectors also include a replicon that functions in E. coli, a gene encoding antibiotic resistance to permit selection of bacteria that harbor recombinant plasmids, and unique restriction sites in nonessential regions of the plasmid to allow insertion of recombinant sequences.

Standard transfection methods are used to produce bacterial, mammalian, yeast or insect cell lines that express large quantities of protein, which are then purified using standard techniques (see, e.g., Colley et al., 1989, J. Biol. Chem., 264:17619-22; Guide to Protein Purification, in Methods in Enzymology, vol. 182 (Deutscher, ed., 1990)). Transformation of eukaryotic and prokaryotic cells are performed according to standard techniques (see, e.g., Morrison, 1977, J. Bacteriol. 132:349-351; Clark-Curtiss & Curtiss, Methods in Enzymology 101:347-362 (Wu et al., eds, 1983)).

Any of the known procedures for introducing foreign nucleotide sequences into host cells may be used. These include the use of calcium phosphate transfection, polybrene, protoplast fusion, electroporation, nucleofection, liposomes, microinjection, naked DNA, plasmid vectors, viral vectors, both episomal and integrative, and any of the other well-known methods for introducing cloned genomic DNA, cDNA, synthetic DNA or other foreign genetic material into a host cell (see, e.g., Sambrook et al., supra). It is only necessary that the particular genetic engineering procedure used be capable of successfully introducing at least one gene into the host cell capable of expressing the DBD nuclease fusion protein.

In embodiments where the DBD nuclease fusion protein contains a CRISPR protein (e.g., dCas9), the methods can include delivering the fusion protein and guide RNA together, e.g., as a complex. For example, the dCas9 nuclease fusion protein described herein and gRNA can be can be overexpressed in a host cell and purified, then complexed with the guide RNA (e.g., in a test tube) to form a ribonucleoprotein (RNP), and delivered to cells. In some embodiments, the dCas9 nuclease fusion protein can be expressed in and purified from bacteria through the use of bacterial dCas9 nuclease fusion protein expression plasmids. For example, His-tagged dCas9 nuclease fusion proteins can be expressed in bacterial cells and then purified using nickel affinity chromatography. The use of RNPs circumvents the necessity of delivering plasmid DNAs encoding the nuclease or the guide, or encoding the nuclease as an mRNA. RNP delivery may also improve specificity, presumably because the half-life of the RNP is shorter and there's no persistent expression of the nuclease and guide (as you'd get from a plasmid). The RNPs can be delivered to the cells in vivo or in vitro, e.g., using lipid-mediated transfection or electroporation. See, e.g., Liang et al. “Rapid and highly efficient mammalian cell engineering via Cas9 protein transfection.” Journal of biotechnology 208 (2015): 44-53; Zuris, John A., et al. “Cationic lipid-mediated delivery of proteins enables efficient protein-based genome editing in vitro and in vivo.” Nature biotechnology 33.1 (2015): 73-80; Kim et al. “Highly efficient RNA-guided genome editing in human cells via delivery of purified Cas9 ribonucleoproteins.” Genome research 24.6 (2014): 1012-1019.

Also provided herein are nucleic acids encoding the fusion proteins, as well as cells, tissues, and transgenic animals comprising the nucleic acids and optionally expressing the fusion proteins. Any nucleic acid construct capable of directing expression and/or which can transfer sequences to target cells can be used to administer the nucleic acid sequences described herein encoding either the exogenous nucleic acid sequence to be inserted within the target site or the zinc finger nuclease/dCas9 fusion proteins. Nucleic acid sequences described herein can be delivered to cells with vector delivery systems, including viral vector delivery systems comprising DNA and RNA viruses, which have either episomal or integrated genomes after delivery to the cell.

The term “vector” as used herein refers to nucleic acid molecules, usually double-stranded DNA, which may have inserted into it another nucleic acid molecule, such as a sequence encoding a nuclease fusion protein. The vector is used to transport the inserted nucleic acid molecule into a suitable host cell. A vector may contain the necessary elements that permit transcribing the inserted nucleic acid molecule, and translating the transcript into a polypeptide. Once in the host cell, the vector may for instance replicate independently of, or coincidental with, the host chromosomal DNA, and several copies of the vector and its inserted nucleic acid molecule may be generated. The term “vector” may thus also be defined as a gene delivery vehicle that facilitates gene transfer into a target cell. This definition includes both non-viral and viral vectors. Alternatively, gene delivery systems can be used to combine viral and non-viral components, such as nanoparticles or virosomes (Yamada et al. (2003) Nat Biotechnol. 21, 885-890). Non-viral vectors include but are not limited to cationic lipids, liposomes, nanoparticles, PEG, PEI, etc. Viral vectors are derived from viruses including but not limited to: retrovirus, lentivirus, adeno-associated virus, adenovirus, herpesvirus, hepatitis virus or the like. Typically, but not necessarily, viral vectors are replication-deficient as they have lost the ability to propagate in a given cell since viral genes essential for replication have been eliminated from the viral vector.

The use of RNA or DNA viral based systems for the delivery of nucleic acids takes advantage of highly evolved processes for targeting a virus to specific cells in the body and trafficking the viral payload to the nucleus. Viral vectors can be derived from lentivirus, adeno-associated virus, adenovirus, retroviruses and antiviruses. Conventional viral based systems for the delivery of nucleic acid sequences could include retroviral, lentiviral, adenoviral, adeno-associated, herpes simplex virus, and TMV-like viral vectors for gene transfer. Integration in the host genome is possible with the retrovirus, lentivirus, and adeno-associated virus gene transfer methods, often resulting in long term expression of the inserted transgene. Additionally, high transduction efficiencies have been observed in many different cell types and target tissues.

Retroviruses and antiviruses are RNA viruses that have the ability to insert their genes into host cell chromosomes after infection. Retroviral and lentiviral vectors have been developed that lack the genes encoding viral proteins, but retain the ability to infect cells and insert their genes into the chromosomes of the target cell (Miller (1990) Mol Cell Biol. 10, 4239-4242; Naldini et al. (1996) Science 272, 263-267; VandenDriessche et al., (1999) Proc Natl Acad Sci USA. 96, 10379-10384. The difference between a lentiviral and a classical Moloney-murine leukemia-virus (MLV) based retroviral vector is that lentiviral vectors can transduce both dividing and non-dividing cells whereas MLV-based retroviral vectors can only transduce dividing cells.

Adenoviral vectors are designed to be administered directly to a living subject. Unlike retroviral vectors, most of the adenoviral vector genomes do not integrate into the chromosome of the host cell. Instead, genes introduced into cells using adenoviral vectors are maintained in the nucleus as an extrachromosomal element (episome) that persists for an extended period of time. Adenoviral vectors will transduce dividing and nondividing cells in many different tissues (Chuah et al. (2003) Blood. 101, 1734-1743). Another viral vector is derived from the herpes simplex virus, a large, double-stranded DNA virus. Recombinant forms of the vaccinia virus, another dsDNA virus, can accommodate large inserts and are generated by homologous recombination.

Adeno-associated virus (AAV) is a small ssDNA virus which infects humans and some other primate species, not known to cause disease and consequently causing only a very mild immune response. AAV can infect both dividing and non-dividing cells and may incorporate its genome into that of the host cell. These features make AAV a very attractive candidate for creating viral vectors for gene therapy, although the cloning capacity of the vector is relatively limited. In a specific embodiment described herein, the vector used is therefore derived from adeno associated virus.

Zinc finger nuclease or dCas9 nuclease fusions with an associated gRNA or crRNA-tracrRNA complex can also be delivered directly as isolated protein or isolated ribonucleoprotein complexes, respectively. The nuclease fusion proteins described herein can be delivered to cells by conventional protein transduction methods known in the art. In specific embodiments, one or more Nuclear Localization Signals (NLS) or protein transduction domains (e.g., penetratin or transportan) can be optionally added to the fusion protein. Such methods are described, for example by Liu, J. et al, Molecular Therapy-Nucleic Acids (2015) 4, e232 and Gaj, T. et al, ACS Chem. Biol. 2014, 9, 1662-1667.

In other embodiments, the nuclease fusion proteins include a cell-penetrating peptide sequence that facilitates delivery to the intracellular space, e.g., HIV-derived TAT peptide or hCT derived cell-penetrating peptides, see, e.g., Caron et al., (2001) Mol Ther. 3(3):310-8; Langel, Cell-Penetrating Peptides: Processes and Applications (CRC Press, Boca Raton Fla. 2002); El-Andaloussi et al., (2005) Curr Pharm Des. 11(28):3597-611; and Deshayes et al., (2005) Cell Mol Life Sci. 62(16):1839-49.

Cell penetrating peptides (CPPs) are short peptides that facilitate the movement of a wide range of biomolecules across the cell membrane into the cytoplasm or other organelles, e.g. the mitochondria and the nucleus. Examples of molecules that can be delivered by CPPs include therapeutic drugs, plasmid DNA, oligonucleotides, siRNA, peptide-nucleic acid (PNA), proteins, peptides, nanoparticles, and liposomes. CPPs are generally 30 amino acids or less, are derived from naturally or non-naturally occurring protein or chimeric sequences, and contain either a high relative abundance of positively charged amino acids, e.g. lysine or arginine, or an alternating pattern of polar and non-polar amino acids. CPPs that are commonly used in the art include Tat (Frankel et al., (1988) Cell. 55:1189-1193, Vives et al., (1997) J. Biol. Chem. 272:16010-16017), penetratin (Derossi et al., (1994) J. Biol. Chem. 269:10444-10450), polyarginine peptide sequences (Wender et al., (2000) Proc. Natl. Acad. Sci. USA 97:13003-13008, Futaki et al., (2001) J. Biol. Chem. 276:5836-5840), and transportan (Pooga et al., (1998) Nat. Biotechnol. 16:857-861).

CPPs can be linked with their cargo through covalent or non-covalent strategies. Methods for covalently joining a CPP and its cargo are known in the art, e.g. chemical cross-linking (Stetsenko et al., (2000) J. Org. Chem. 65:4900-4909, Gait et al. (2003) Cell. Mol. Life. Sci. 60:844-853) or cloning a fusion protein (Nagahara et al., (1998) Nat. Med. 4:1449-1453). Non-covalent coupling between the cargo and short amphipathic CPPs comprising polar and non-polar domains is established through electrostatic and hydrophobic interactions.

CPPs have been utilized in the art to deliver potentially therapeutic biomolecules into cells. Examples include cyclosporine linked to polyarginine for immunosuppression (Rothbard et al., (2000) Nature Medicine 6(11):1253-1257), siRNA against cyclin B1 linked to a CPP called MPG for inhibiting tumorigenesis (Crombez et al., (2007) Biochem Soc. Trans. 35:44-46), tumor suppressor p53 peptides linked to CPPs to reduce cancer cell growth (Takenobu et al., (2002) Mol. Cancer Ther. 1(12):1043-1049, Snyder et al., (2004) PLoS Biol. 2:E36), and dominant negative forms of Ras or phosphoinositol 3 kinase (PI3K) fused to Tat to treat asthma (Myou et al., (2003) J. Immunol. 171:4399-4405).

CPPs have been utilized in the art to transport contrast agents into cells for imaging and biosensing applications. For example, green fluorescent protein (GFP) attached to Tat has been used to label cancer cells (Shokolenko et al., (2005) DNA Repair 4(4):511-518). Tat conjugated to quantum dots have been used to successfully cross the blood-brain barrier for visualization of the rat brain (Santra et al., (2005) Chem. Commun. 3144-3146). CPPs have also been combined with magnetic resonance imaging techniques for cell imaging (Liu et al., (2006) Biochem. and Biophys. Res. Comm. 347(1):133-140). See also Ramsey and Flynn, Pharmacol Ther. 2015 Jul. 22. pii: S0163-7258(15)00141-2.

In some embodiments, the nuclease fusion proteins include a moiety that has a high affinity for a ligand, for example GST, FLAG or hexahistidine sequences. Such affinity tags can facilitate the purification of recombinant nuclease fusion proteins.

Also provided herein are compositions and kits comprising the nuclease fusion proteins described herein. In some embodiments where the DNA binding domain is dCas9, the kits include the fusion proteins and a c guide RNA (i.e., a guide RNA that binds to the protein and directs it to a target sequence appropriate for that protein). In some embodiments, the kits also include labeled detector DNA, e.g., for use in a method of detecting a target ssDNA or dsDNA. Labeled detector DNAs are known in the art, e.g., as described in US20170362644; East-Seletsky et al., Nature. 2016 Oct. 13; 538(7624): 270-273; Gootenberg et al., Science. 2017 Apr. 28; 356(6336): 438-442, and WO2017219027A1, and can include labeled detector DNAs comprising a fluorescence resonance energy transfer (FRET) pair or a quencher/fluorophore pair, or both. The kits can also include one or more additional reagents, e.g., additional enzymes (such as RNA polymerases) and buffers, e.g., for use in a method described herein.

The present invention is additionally described by way of the following illustrative, non-limiting Examples that provide a better understanding of the present invention and of its many advantages.

### EXAMPLES

The following Examples illustrate some embodiments and aspects of the invention. It will be apparent to those skilled in the relevant art that various modifications, additions, substitutions, and the like can be performed without altering the spirit or scope of the invention, and such modifications and variations are encompassed within the scope of the invention as defined in the claims which follow. The following Examples do not in any way limit the invention.

### Example 1

### Development of Targetable Nucleases that can Induce DSBs with 3′ Overhangs

To develop targetable nucleases that can induce DSBs with 3′ overhangs, nuclease domains derived from Type IIS restriction enzymes that were believed to create such overhangs were identified. Type IIS restriction enzymes have distinct DNA-binding and nuclease domains, which can be separated by a DNA methytransferase domain. In principle, this architecture enabled the nuclease domain to be potentially separated from the native DNA-binding domain and fused to other customizable DNA-binding scaffolds. For example, previously described engineered zinc finger nucleases consisted of the nuclease domain from the Type IIS FokI restriction enzyme fused to an array of engineered zinc fingers. Similarly, this FokI nuclease domain has also been fused to transcription activator-like effector (TALE) domain arrays and catalytically inactive Cas9 (dead Cas9 or dCas9) to create TALE nucleases (TALENs) and FokI-dCas9 (also referred to as fCas9 or RNA-guided FokI Nucleases (RFNs)) nucleases, respectively. It was believed that no nuclease domain from a TypeIIS enzyme that generated 3′ overhang DSBs had been separated from its native DNA binding domain and fused to a heterologous domain. Creating such fusions was hypothesized to be desirable because models of homology-directed repair suggested that double-strand breaks were processed to 3′ overhangs by DNA repair machinery in order to initiate such repair. This further suggested that targetable nucleases that induce 3′ overhangs might be more efficient at inducing homology-directed repair than nucleases that induce 5′ overhangs (e.g., FokI-based ZFNs, TALENs, FokI-dCas9/fCas9/RFNs, CRISPR-Cpfl nucleases) or blunt ends (e.g., CRISPR-Cas9 nucleases). However, determining whether 3′ overhangs were actually more efficient for HDR has been difficult to prove because performing the necessary direct comparisons was challenging due to the difficulty in creating different overhangs at the same sequence.

To identify a potential nuclease domain that could be used to create 3′ overhang DSBs, a search of the published literature and the REBASE database (Roberts, R. J. et al. Nucleic Acids Res. (2015)) was performed. This search identified a large number of Type IIS restriction enzymes that have been reported to induce DSBs with 3′ overhangs (Table 1).

TABLE 1
Type II Restriction Enzymes that Leave a 3′ Overhang
Nuclease domain size is indicated where known. 3′ overhang size
is indicated. Those indicated as fragment are where the cleavage
of DNA is staggered by the enzyme and will result in the excision
of a fragment of varying size with 3′ overhangs of size indicated.
Enzymes selected for further investigation are bolded. FokI
(italicized) is included in the table for reference.

| Enzyme | 3′ Overhang | Size of Nuclease Domain |
|---|---|---|
| CjePI | 6 nt, fragment | |
| CjeI | 6 nt, fragment | |
| Arsl | 5 nt, fragment | |
| Bsp241 | 5 nt, fragment | |
| HaeIV | 5 nt, fragment | |
| Tstl | 5 nt, fragment | |
| Alol | 5-8 nt, fragment | 405aa |
| Hin4I | 5-6 nt, fragment | |
| BaeI | 5 nt, fragment | 249aa |
| BarI | 5 nt, fragment | |
| BplI | 5 nt, fragment | |
| CjePI | 5 nt, fragment | |
| FalI | 5 nt, fragment | |
| PpiI | 5 nt, fragment | |
| PsrI | 5 nt, fragment | |
| FokI | 4nt 5′ overhang | 206aa |
| BsaXI | 3 nt, Fragment | |
| RleAI | 3 nt | |
| WviI | 3 nt | |
| SdeOSI | 2 nt, fragment | |
| AcuI | 2 nt | |
| ApyPI | 2 nt | |
| AQuIII | 2 nt | |
| AquIV | 2 nt | |
| Bce83I | 2 nt | |
| BfuI | 2 nt | |
| BpmI | 2 nt | |
| BpuEI | 2 nt | |
| BsbI | 2 nt | |
| Bse3DI | 2 nt | |
| BseGI | 2 nt | |
| BseMI | 2 nt | |
| BseMII | 2 nt | |
| BsgI | 2 nt | |
| BtsI | 2 nt | |
| CdpI | 2 nt | |
| CstMI | 2 nt | |
| DraRI | 2 nt | |
| EciI | 2 nt | |
| CsuI | 2 nt | |
| HauII | 2 nt | |
| MaqI | 2 nt | |
| MmeI | 2 nt | |
| NaCI | 2 nt | |
| PlaDI | 2 nt | |
| RceI | 2 nt | |
| RpaBI | 2 nt | |
| RpaI | 2 nt | |
| SdeAI | 2 nt | |
| TaqII | 2 nt | |
| TsoI | 2 nt | |
| AsuHPI | 1 nt | |
| BeiVI | 1 nt | |
| BfiI | 1 nt | |
| BmiI | 1 nt | |
| BmuI | 1 nt | |
| BsuI | 1 nt | |
| Hin4II | 1 nt | |
| HphI | 1 nt | |
| MboII | 1 nt | |
| NcuI | 1 nt | |

Because a nuclease domain that was dimerization-dependent (analogous to the FokI nuclease domain) would be optimal, the resulting list of enzymes was further limited by identifying those for which evidence of dimerization-dependent activity exists in the published literature. The resulting narrowed list consisted of five restrictions enzymes (AcuI, AloI, BpmI, BaeI, and MmeI) that include DSBs with variable length 3′ overhands (Table 1, bolded). Using available amino acid sequence data in the NCBI protein database and knowledge of the typical structure of IIS enzymes, we predicted putative nuclease domains for the five restriction enzymes, AcuI, AloI, BpmI, BaeI, and MmeI (FIGS. 3A-E).

To test whether these defined or putatively defined 3′ overhang nuclease domains would work when fused to a heterologous sequence-specific DNA binding domain and to attempt to engineer targetable nucleases that leave 3′ overhangs, each of the five nuclease domains identified from AcuI, AloI, BpmI, BaeI, and MmeI were fused to dCas9 derived from Streptococcus pyogenes. Two types of fusions were constructed for each of the five nuclease domains: one in which the nuclease domain was fused to the amino-terminal end of dCas9 and the other in which the nuclease domain was fused to the carboxy-terminal end of dCas9. For both types of fusions, a linker of sequence GGGGS (G4S) (SEQ ID NO: 3) was used to connect these nuclease domains to dCas9. It was envisioned that, like FokI nuclease domain fusions to dCas9, dimers of some of the constructed fusions could only mediate sequence-specific DNA cleavage when bound to target sites composed of two “half-sites” (each bound by one dCas9 monomer domain) in the correct orientation and with a certain defined length ‘spacer’ sequence between them.

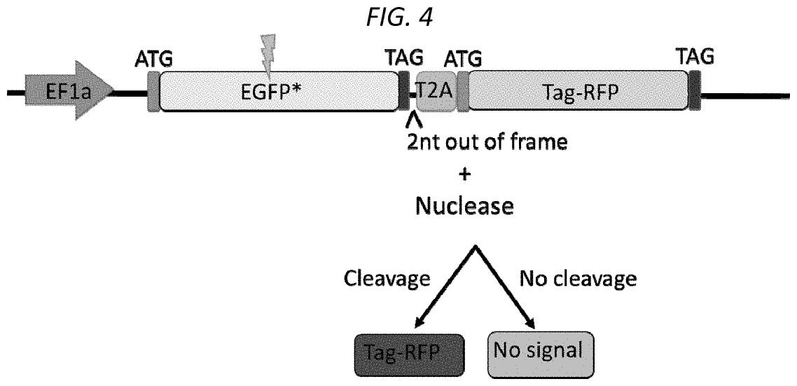

To determine the specific half-site orientations and spacings that would enable efficient cleavage by the ten different fusions, a previously described human cell-based RFP gain-of-expression reporter assay was used (Certo, M., et al. Nature Methods (2012)). This assay used an engineered human U2OS cell line that harbors a single copy of a constitutively expressed EGFP*-T2A-RFP fusion reporter gene (the cell line is named the U2OS.traffic light reporter cell line or U2OS.TLR). The EGFP* gene had a single bp nonsense mutation and the RFP reporter gene was 2 nucleotides out of frame with the EGFP* mutant reporter gene and therefore the U2OS.TLR cells were EGFP-negative and RFP-negative. If a site-specific nuclease targeted to the EGFP* reporter gene was able to cleave its target site, subsequent repair by non-homologous end-joining led to the induction of variable-length indel mutations, a subset of which could have brought the RFP reporter gene in frame with the EGFP* gene reading frame, resulting in cells that are then RFP-positive. Thus, the percentage of RFP-positive cells induced in a population of U2OS.TLR cells transfected with a nucleic acid encoding a given targeted nuclease served as an indirect measure of the efficiency of cleavage by that nuclease (FIG. 4).

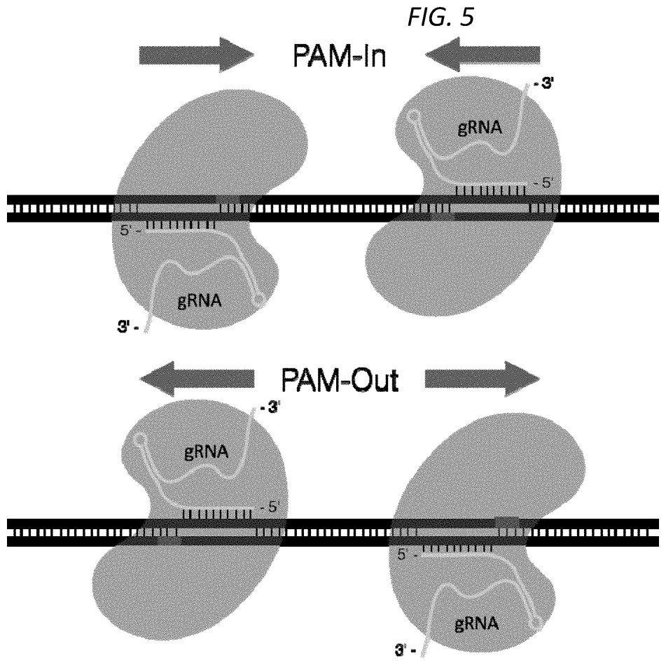

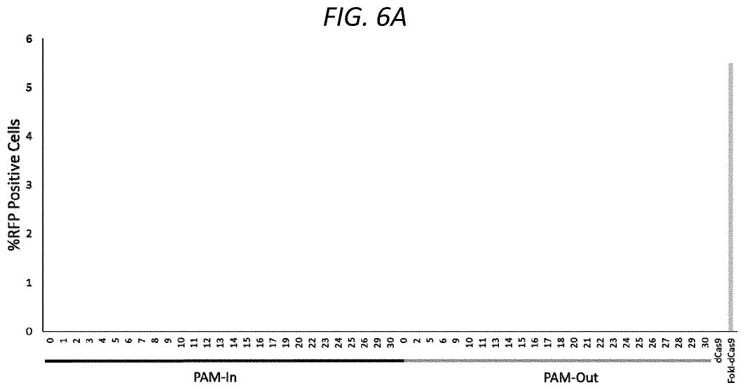

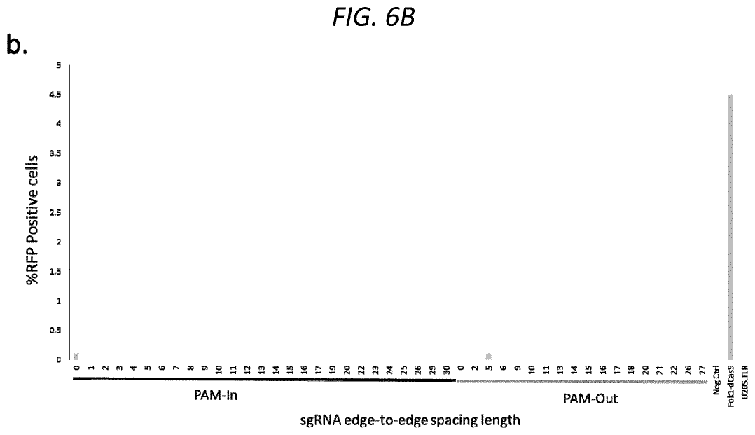

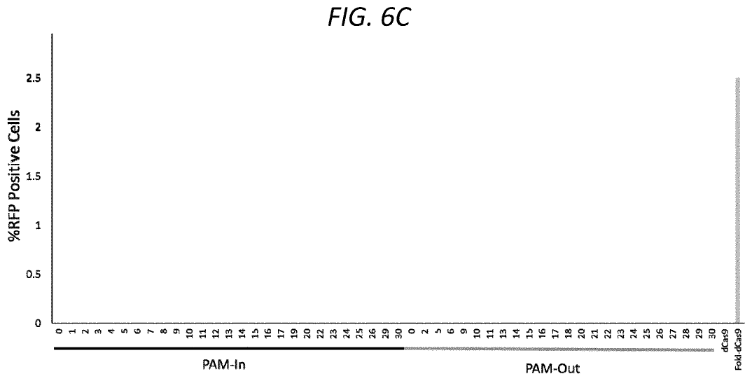

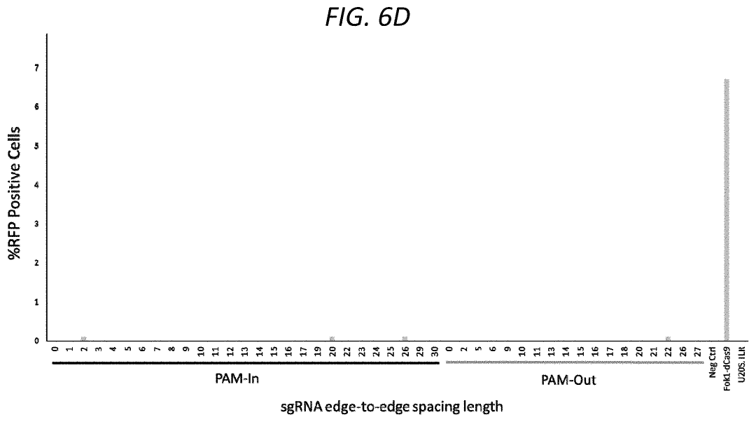

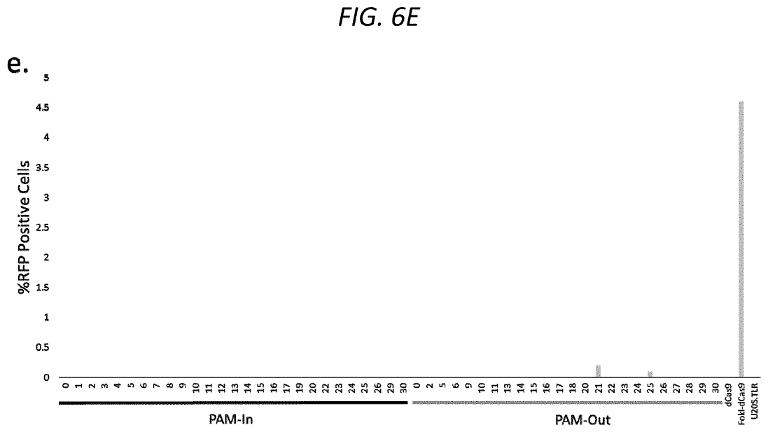

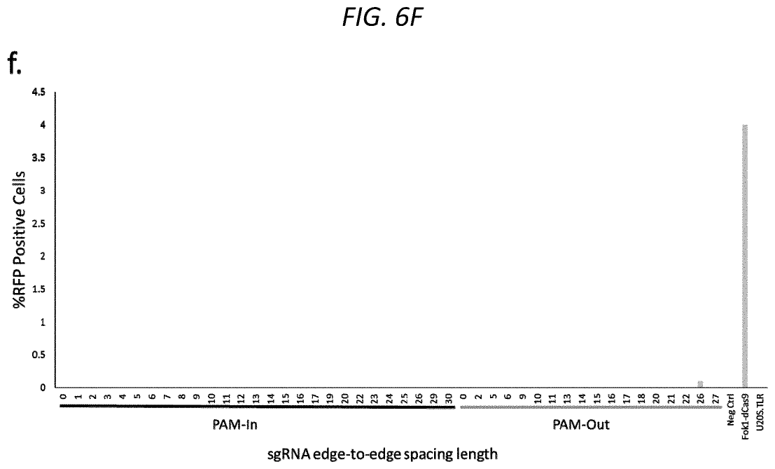

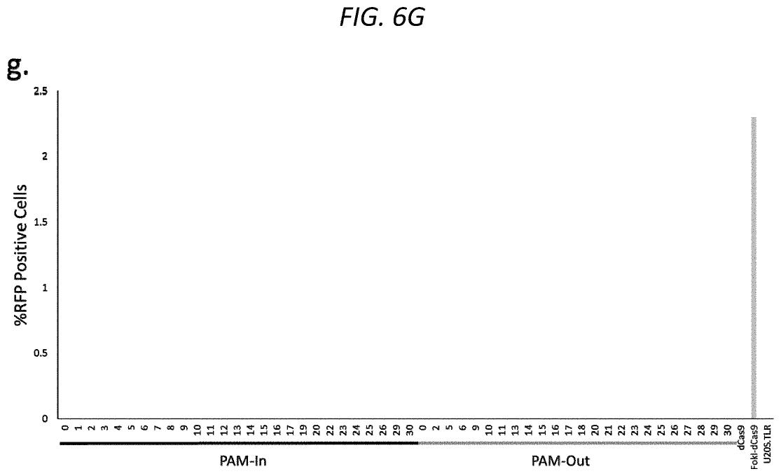

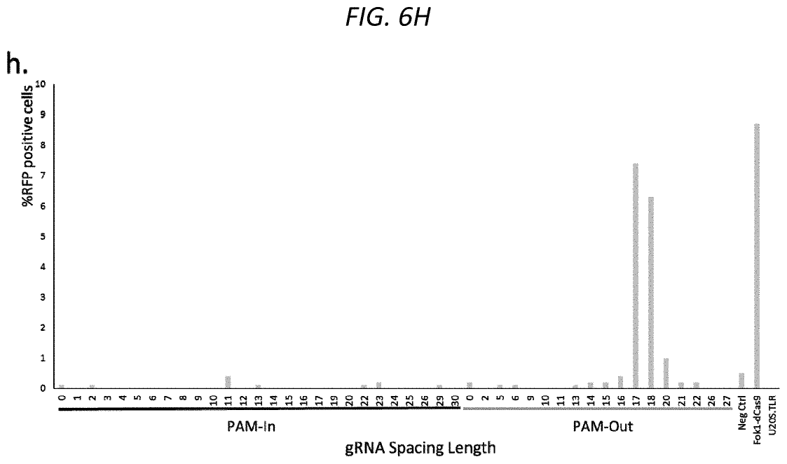

To determine whether the various nuclease-dCas9 fusions were capable of cleaving specific target sites in human cells, various pairs of gRNAs were designed that would target two nuclease/dCas9 molecules to “half-sites” in EGFP arranged in various orientations and spacings relative to each other. The two half-sites targeted by each of these gRNA pairs were oriented such that both of their PAM sequences were either directly adjacent to the spacer sequence (the “PAM-in” orientation) or positioned at the outer boundaries of the full-length target site (the “PAM-out” orientation) (FIG. 5). The spacer sequence (between the two half-sites) was also varied in length from 0 to 31 hp for both the PAM-in and PAM-out orientations. In tests of the various nuclease-dCas9 fusions at these different target sites, there was no evidence of robust nuclease activity (as judged by an increase in the percentage of RFP-positive U2OS.TLR cells) with any of the gRNA pairs that were tested with the dCas9-AcuI, AloI-dCas9, dCas9-AloI, BpmI-dCas9, dCas9-BpmI, BaeI-dCas9, dCas9-BaeI, dCas9-MmeI, and MmeI-dCas9 fusions (fusions were named according to the order of the domains within the fusion going from amino-terminus to carboxy-terminus; FIG. 6A-J). The AcuI-dCas9 nuclease did not show activity with gRNA pairs that orient the two half sites in the PAM-in orientation but did show robust activity with gRNA pairs that orient the half-sites in the PAM-out orientation with spacings of 17, 18 and 20 bps (note that no spacing of 19 bps was tested) (FIG. 6H). (Note that this activity profile differed from that observed with FokI-dCas9 fusions which had activity over a broader range of spacings from 13 to 18 bps and 26 bps between half-sites oriented in the PAM-out orientation—see Tsai et al., Nat Biotechnol. 2014).

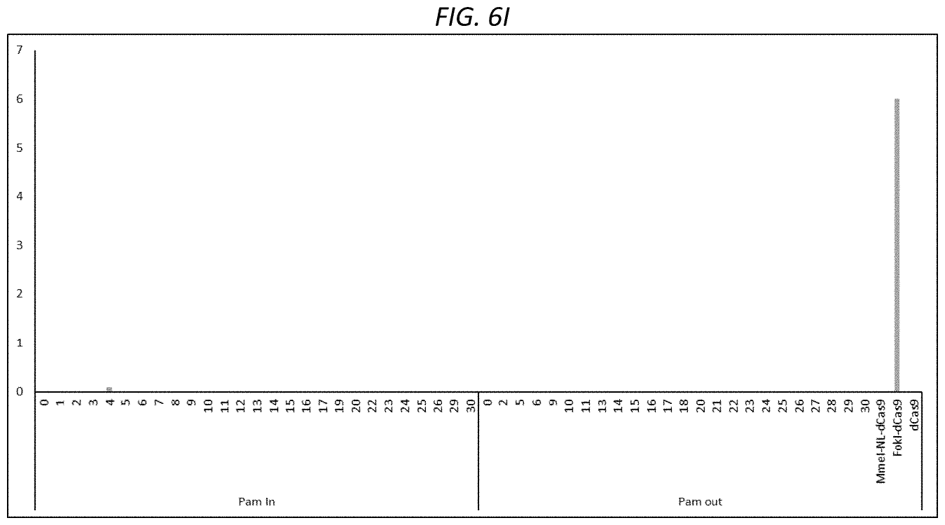

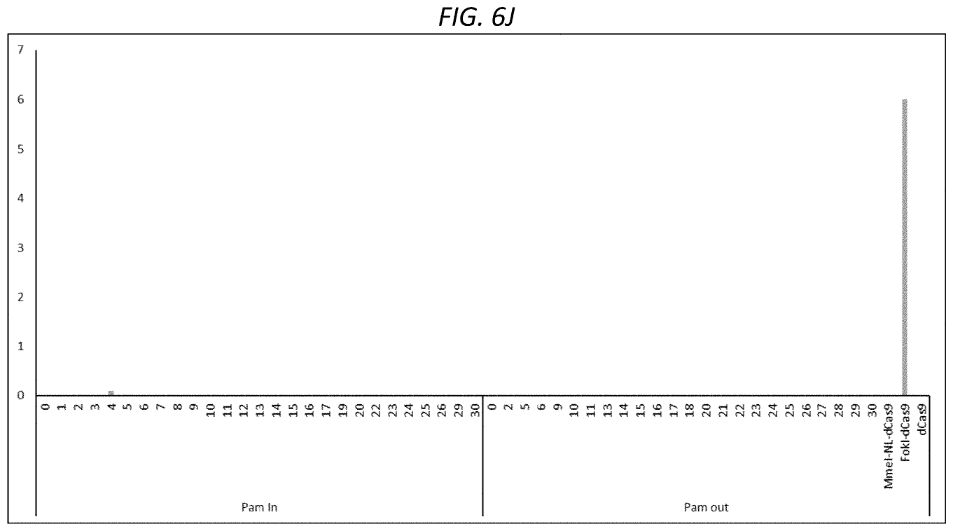

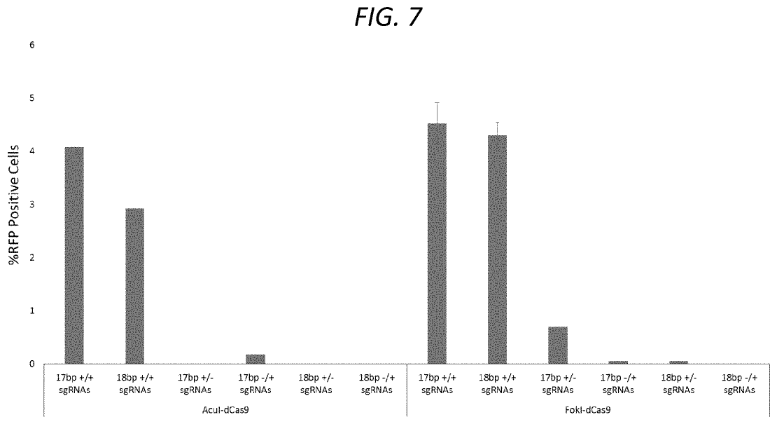

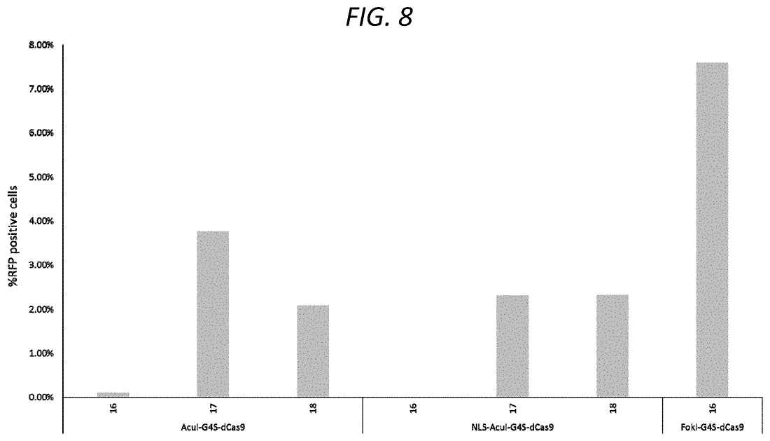

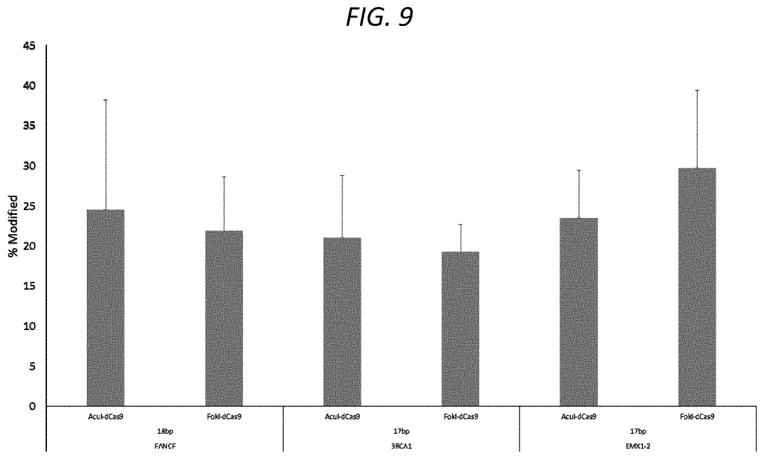

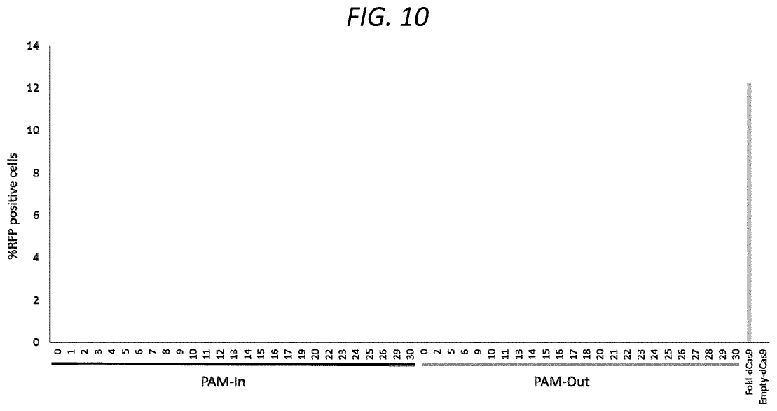

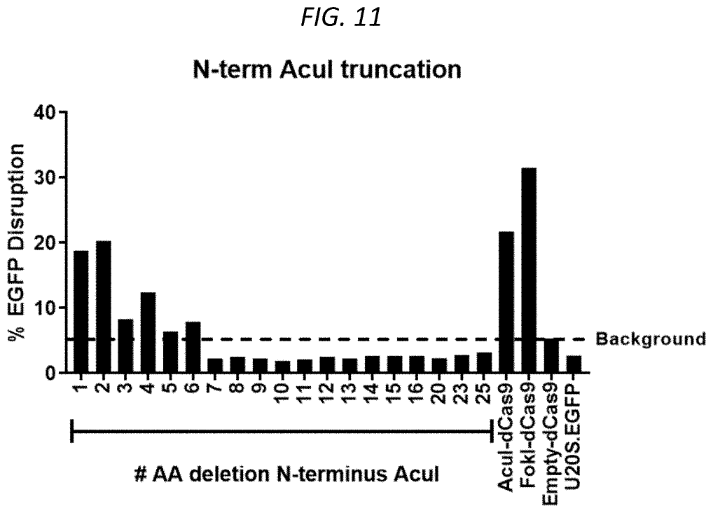

Additional experiments with the AcuI-dCas9 fusion demonstrated that, as is observed with the previously described FokI-dCas9 fusion, efficient cleavage at target sites with 17 or 18 bp spacings required both gRNAs in a pair (i.e., that cleavage was not observed when only one gRNA is provided) (FIG. 7); this suggested that dimerization of AcuI nuclease domains on the target site was required for efficient cleavage. Addition of a nuclear localization signal (NLS) to the nuclease fusions neither improved nor reduced the activity of the AcuI-dCas9 fusion (FIG. 8). In addition, the activities of the AcuI-dCas9 fusion and the FokI-dCas9 fusion were directly compared using the same pairs of gRNAs for the same sites (with spacings of 17 and 18 bps) and it was shown that their activities were comparable (as judged by the RFP gain-of-function assay as well as the well-established T7 Endonuclease I (T7EI) assays performed on multiple endogenous sites; FIG. 8 and FIG. 9 respectively). Finally, a more truncated version of the AcuI nuclease domain (amino acids 26 to 199 from AcuI) was evaluated. AcuI-dCas9 fusions made with this shortened domain were not functional on any target sites tested (0-31 bp spacers in either the PAM-In or PAM-out orientation) (FIG. 10). Additional analysis of a series of truncation mutants in which variable numbers of amino acids (ranging from 1 to 25) were deleted from the amino-terminal end of the AcuI nuclease domain present in the AcuI-dCas9 fusion showed that amino acid positions 1 and 2 were dispensable for function but that deletion of more than these amino acids leads to substantial or complete loss of genome editing activity (FIG. 11).

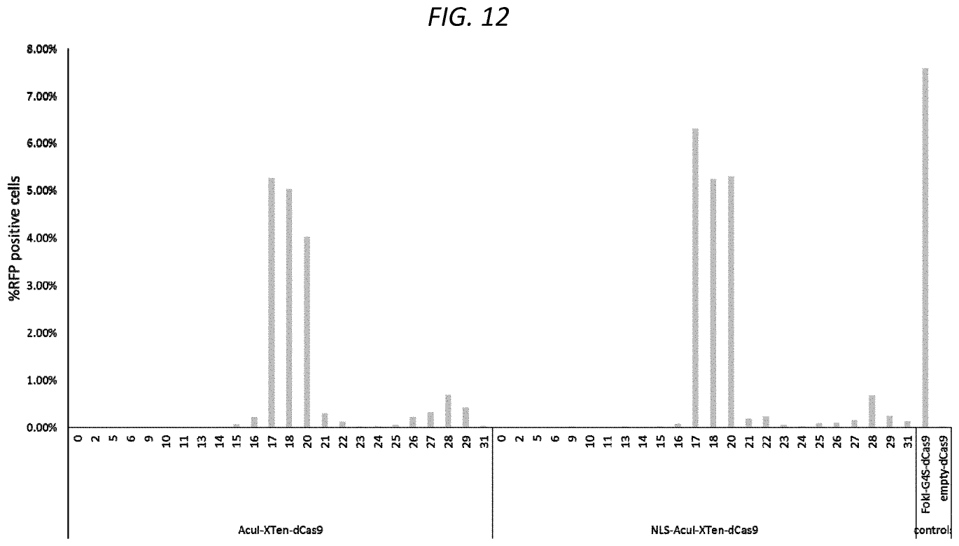

It was next determined whether varying the amino acid composition and length of the linker between the AcuI nuclease domain and dCas9 might alter the profile of sites that could be cleaved by the AcuI-dCas9 fusion, in particular, whether sites with different spacer lengths between the half-sites might be cleaved. To do this, the original AcuI-dCas9 fusion (with a flexible G4S linker) was compared with a new XTEN derivative harboring the extended-conformation linker (Guilinger, J., et al. Nature Biotechnology (2014)). The AcuI-dCas9 fusion with an XTEN linker showed generally higher activities than the original fusion at sites with 17, 18, and 20 bp spacers with its greatest effect apparent on the 20 bp spacer site (FIG. 12). As with the original AcuI-dCas9 fusion, the addition of an NLS to the XTEN linker fusion nuclease did not substantially increase or decrease activity (FIG. 12).

### Example 2

### Comparison of HDR Efficiencies Between FokI-dCas9 Fusions and AcuI-dCas9 Fusions

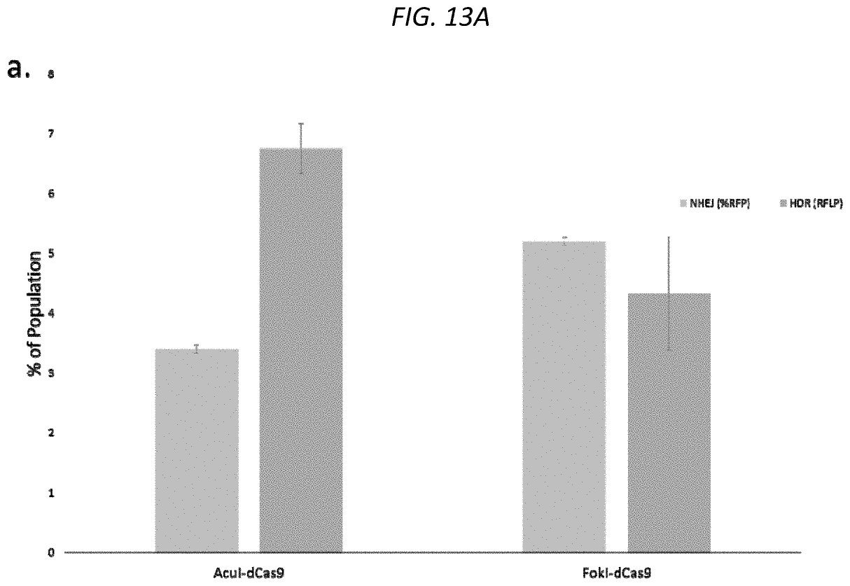

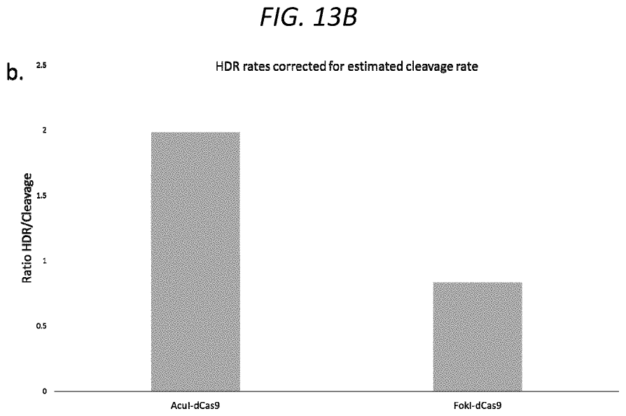

Having established that AcuI-dCas9 fusions was able to site-specifically cleave DNA and induce indel mutations, next, it was investigated whether the 3′ overhangs induced by these fusions might better stimulate HDR events than 5′ overhangs induced at the same sites by FokI-dCas9 fusions. Because both AcuI-dCas9 and FokI-dCas9 fusions were able to cleave target sites composed of half-sites with 17 bp spacers, this enabled the first direct comparison (on the exact same target sites) of the HDR-inducing abilities of nucleases that should generate DSBs with 5′ overhangs (FokI-dCas9 fusion) with those that should generate DSBs with 3′ overhangs (AcuI-dCas9 fusion). In an initial experiment, this comparison was performed on a target site in a constitutively expressed EGFP gene that was integrated in single copy in a human U2OS cell line (named U2OS.EGFP). This target site had a 17 bp spacer between two half-sites targetable by a pair of gRNAs with dCas9, which were oriented in the PAM-out configuration. Using targeted amplicon sequencing, both the frequencies of NHEJ-mediated sequence indels induced at the EGFP gene site by FokI-dCas9 or AcuI-dCas9 fusions and the frequencies of insertion of a 30 BamHI restriction site (GGATCC) via HDR by FokI-dCas9 or AcuI-dCas9 in the presence of a single-stranded oligodeoxynucleotide (ssODN) donor molecule were examined. This experiment demonstrated that although the AcuI-dCas9 enzyme was less efficient at inducing indel mutations than FokI-dCas9, it was more efficient at inducing HDR-mediated alterations (FIG. 13a).

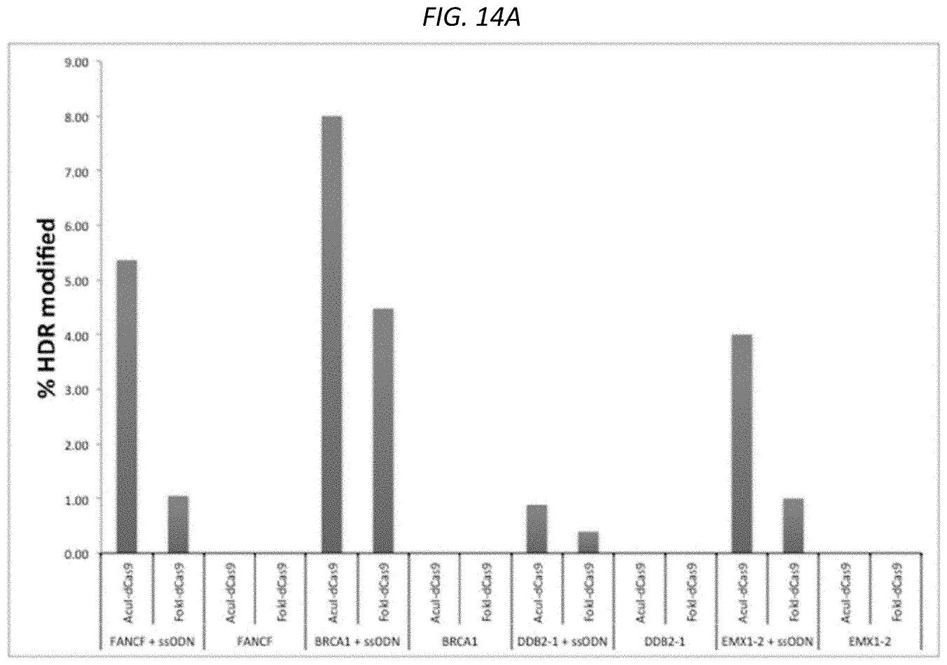

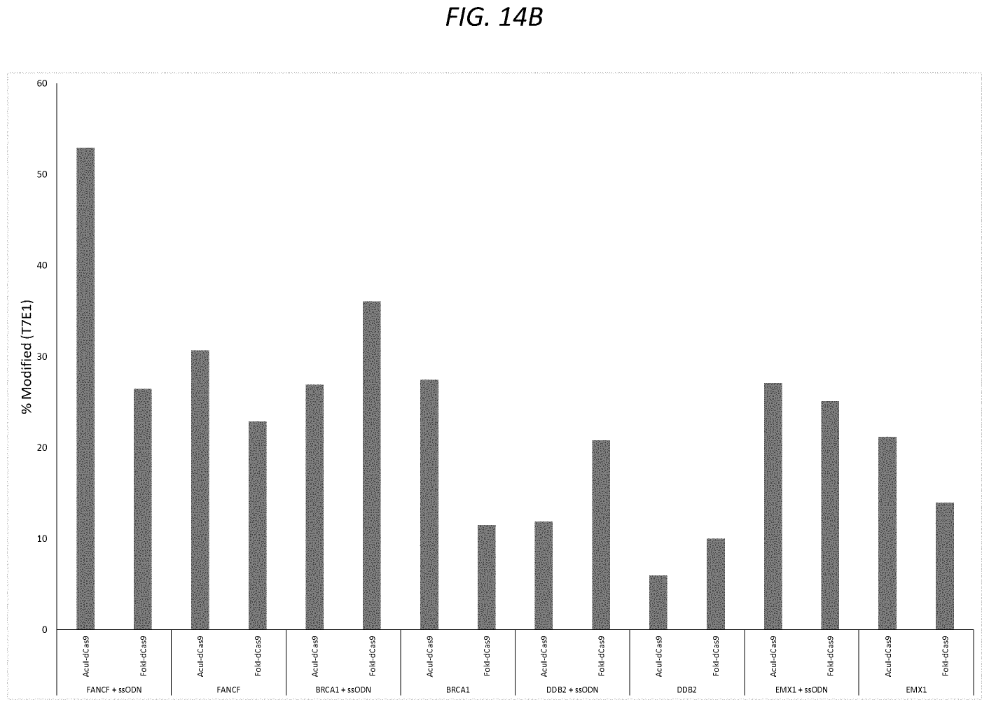

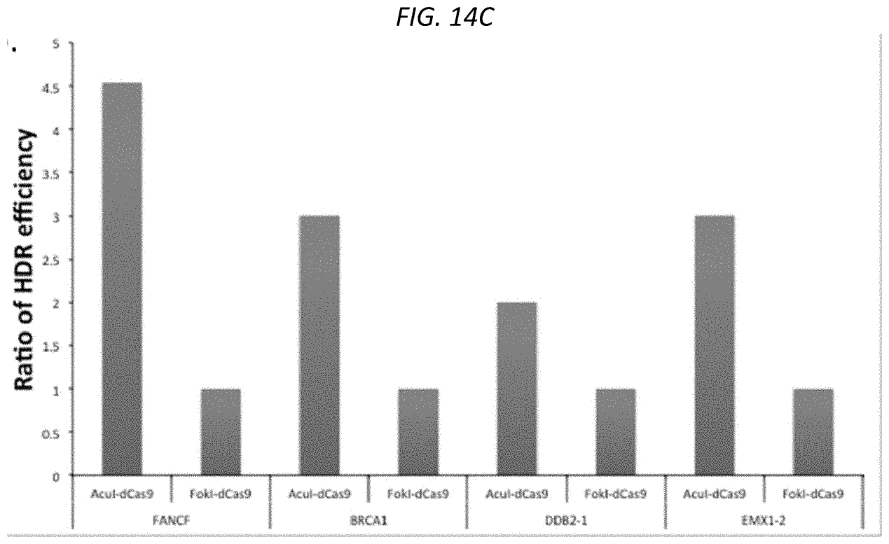

Another way of representing this difference was to examine the ratio of the HDR-mediated alteration efficiency to the NHEJ-mediated indel efficiency, which corrected for the relative cleavage activity of the fusion on the site. By this measure, the AcuI-dCas9 fusion outperformed the FokI-dCas9 fusion by 2-fold (FIG. 13b). The abilities of AcuI-dCas9 and FokI-dCas9 to induce HDR events were compared with an ssODN donor on four additional target sites found in endogenous human genes. All four of these sites had spacer lengths of 17 or 18 bps between the half-sites (oriented in the PAM-out configuration) and thus each of these four sites could be targeted by both AcuI-dCas9 and FokI-dCas9 using the same pair of gRNAs. For these comparisons, the overall efficiency of target site alteration was assessed using the T7EI assay, which quantified the sum total of NHEJ-induced indel mutations and HDR-induced insertions of a BamHI restriction site at the nuclease-induced DSB site. The efficiency of HDR-induced insertions was assessed using an RFLP assay, which only quantified the frequency of HDR-mediated BamHI restriction site insertions into the target site (FIGS. 14a and 14b, respectively). For all four target sites, both the efficiency of HDR-induced insertions and the ratio of the efficiency of HDR-induced insertions to the efficiency of overall target site alteration were higher with AcuI-dCas9 than with FokI-dCas9(FIG. 14c). Collectively, these data from an integrated EGFP reporter and from four different endogenous human gene sites provided the first convincing demonstration that 3′ overhangs (generated by AcuI-dCas9 fusions) were more efficient at inducing HDR events than 5′ overhangs (generated by FokI-dCas9 fusions), demonstrating the importance and applications of targetable nucleases that generate 3′ overhang DNA breaks.

### Example 3

### Zinc Finger Array-AcuI Nuclease Domain Fusions

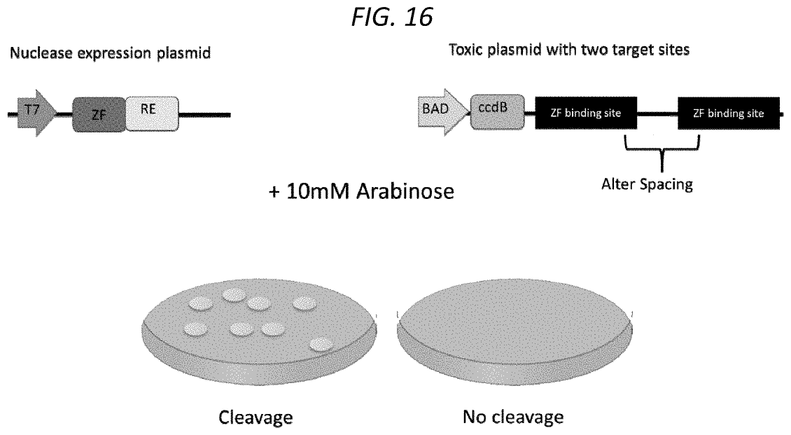

To extend the utility and targetability of the AcuI nuclease domain, it was next determined whether this domain could be fused to engineered zinc finger arrays to create a novel zinc finger nuclease (ZFN) architecture that should induce 3′ overhang DSBs. Standard ZFNs previously described consisted of a FokI nuclease domain (which induces 5′ overhang DSBs) fused to the C-terminal end of a zinc finger array using a linker (e.g., of the form LRGS; FIG. 15). In initial experiments, a ZFN was constructed in which the FokI nuclease domain was replaced with the same AcuI nuclease domain used in the AcuI-dCas9 fusions described above (FIG. 14). This AcuI-based ZFN fusion would be expected to bind and cleave DNA as a dimer, just as the FokI-based ZFNs have been shown to do. To test this, a bacterial cell-based assay was used to assess site-specific nuclease activities (FIG. 16) (Kleinstiver, et al. Nature. (2015)). In this assay, successful cleavage of a particular target site placed within a toxic plasmid by a site-specific nuclease allowed survival of bacterial cells on agar plates.

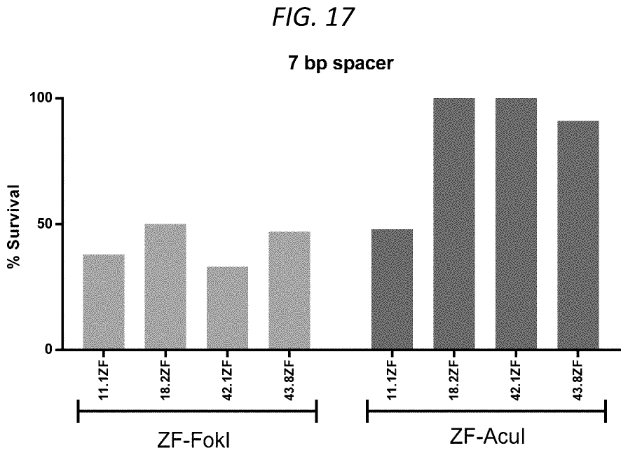

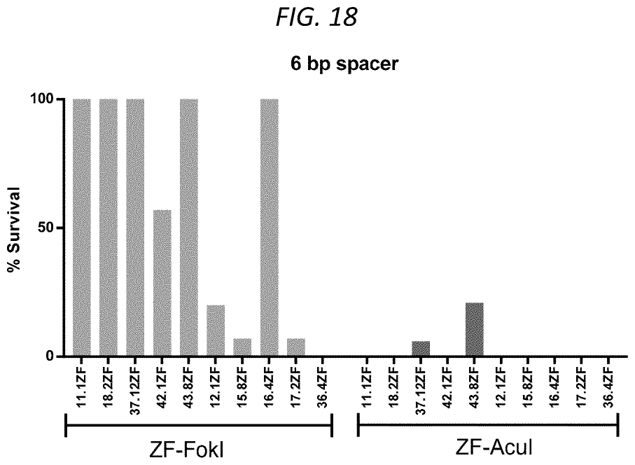

A homodimeric AcuI-based ZFN was tested in the bacterial assay on a variety of target sites bearing spacer lengths ranging from 2 to 11 bps and the most efficient cleavage was found on the site with a 7 bp spacer (FIG. 17). This finding differs from FokI-based ZFNs that possess an LRGS linker, which have previously been shown to efficiently cleave sites with 5 or 6 bp spacers (Wilson et al., Mol. Ther. Nucleic Acids (2013)), a finding that we re-verified using the bacterial cell-based assay (FIG. 18).

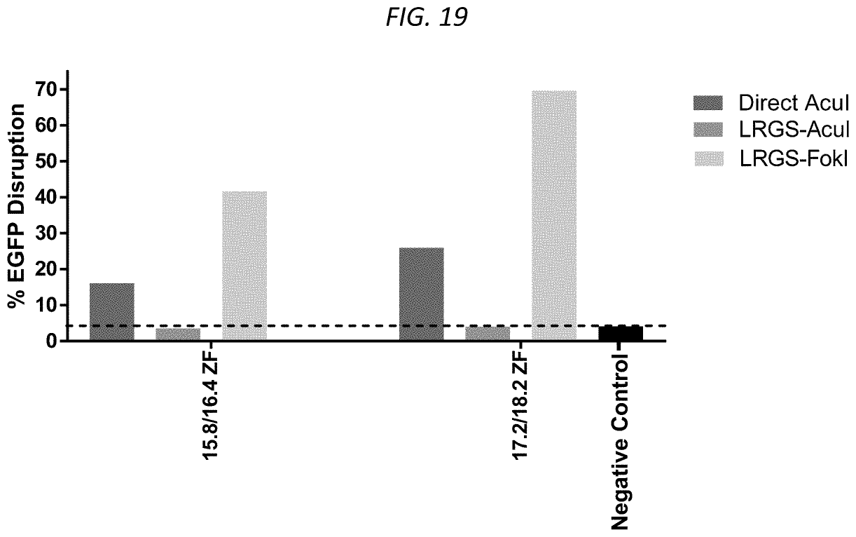

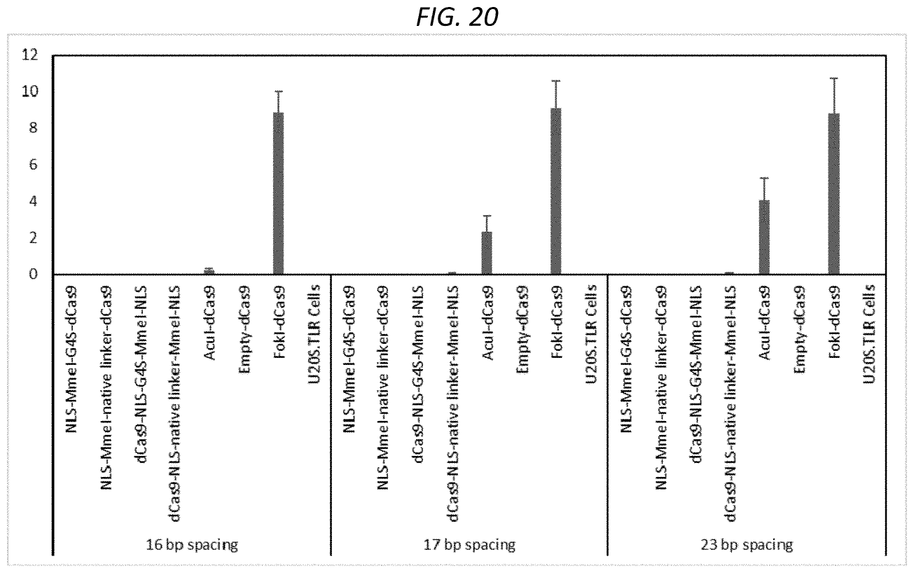

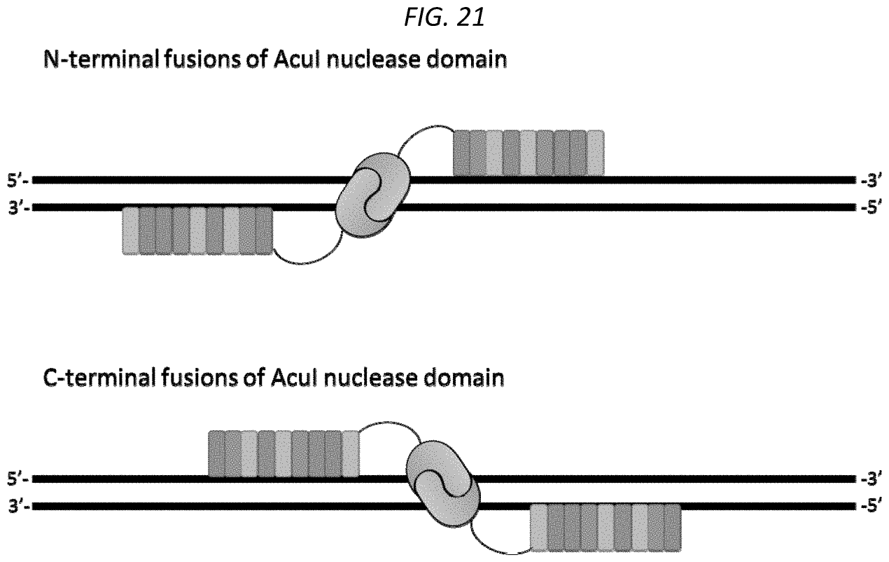

Given the finding in the bacterial cell-based assay that the initial AcuI-based ZFN prototype worked best on target sites in which the half-sites were separated by a 7 bp spacer, this fusion was modified to determine whether it would function on target sites with half-sites separated by a 6 bp spacer. This new fusion architecture comprised a direct fusion of the AcuI nuclease domain to the carboxy-terminal end of a zinc finger array, without any intervening linker. The activities of the original (with an LRGS linker) and the modified (direct fusion with no linker) AcuI-based zinc finger nucleases were tested using the human U2OS cell-based EGFP disruption assay described above (FIG. 11). Two pairs of zinc finger arrays (named 15.8/16.4 and 17.2/18.2) designed to target sequences within the EGFP gene that had 6 bp spacers between the half-sites for each zinc finger array were tested in both AcuI-based nuclease architectures (LRGS linker and no linker). Previously published experiments showed that fusion of these zinc finger arrays to FokI nucleases enabled highly efficient disruption of EGFP activity in human cells (Maeder et al., Mol Cell 2008; PMID: 18657511). Testing of these nucleases showed no increase in EGFP disruption above background (as determined with a negative control) with pairs of AcuI-based fusions harboring an LRGS linker (FIG. 19). However, substantial EGFP disruption was observed with direct fusions that did not have a linker between the zinc finger arrays and the AcuI nuclease domain (FIG. 19), demonstrating that this new architecture could function to cleave sites with a 6 bp spacer in human cells. Positive control fusions of FokI nuclease to the same zinc finger arrays also showed EGFP disruption activity (FIG. 19), consistent with previously published results (Maeder et al., Mol Cell 2008; PMID: 18657511). These results demonstrate that direct fusions of an AcuI nuclease domain to the carboxy-terminus of an engineered zinc finger array can yield ZFNs that can efficiently cleave target DNA in human cells bearing a 6 bp spacer between the zinc finger binding half-sites.

### Example 4

### Materials and Methods for Examples 1-3

Construction of nuclease fusion proteins: Nuclease domains of Type IIS restriction enzymes were fused to the amino-terminal and carboxy-terminal ends of dCas9 and zinc finger arrays via PCR amplification with Phusion polymerase and insertion by Gibson Assembly into digested expression vectors. dCas9 and zinc finger fusions were cloned into a CAG promoter mammalian expression vector and zinc finger fusions were also cloned into a T7 bacterial expression vector. Plasmids encoding multiplex gRNAs were inserted into mammalian expression vector with U6 promoter through standard annealing of oligos and ligation into Csy4-flanked gRNA backbone (SQT1313) digested with BsmBI.

Human Cell Traffic Light Reporter Assay: 200,000 U2OS Traffic Light Reporter (U2OS.TLR) cells were transfected using Lonza 4D nucleofection kits (SE solution, program DN1 00). Cells were analyzed 52 hours post-transfection by flow cytometry to determine the percentage of RFP-positive cells.

Human Cell EGFP Disruption Assay: 200,000 U2OS.EGFP cells were transfected using Lonza 4D nucleofection kits (SE solution, program DN100). Cells were analyzed for cleavage at 52 hours post-transfection by flow cytometry to determine the percentage of EGFP-negative cells.

Quantification of indel mutation rates by T7 Endonuclease I (T7E1) Assay: Genomic DNA of transfected cells was isolated 52 hours post-transfection using Agencourt DNAdvance Genomic DNA Isolation Kit following manufacturer's instructions. PCR amplification of target site was performed with Phusion polymerase generating amplicons ˜800 bp in length using following thermocycler program: 98° C., 30 s; (98° C., 15 s; 58° C., 10 s; 72° C., 15 s)×35; 72° C., 5 min. PCR products were purified using Ampure beads and 200 ng of purified product was denatured, hybridized and treated with 1 ul of T7EI. Mutation rates were calculated as previously described (Reyon et al., Nat Biotechnol. 2012; PMID: 22484455) from data obtained using a Qiaxcel capillary electrophoresis instrument and associated software which quantified areas of the PCR amplified peak and peaks generated from cleavage by T7E1.

Quantification of HDR rates by RFLP: Genomic DNA of transfected cells was isolated 52 hours post-transfection using Agencourt DNAdvance Genomic DNA Isolation Kit following manufacturer's instructions. PCR amplification of target site was performed with Phusion polymerase generating amplicons 800 bp in length using following thermocycler program: 98° C., 30 s; (98° C., 15 s; 58° C., 10 s; 72° C., 15 s)×35; 72° C., 5 min. PCR products were purified using Ampure beads and 200 ng of purified product was treated with BamHI (New England BioLabs). HDR rates were calculated from data obtained using a Qiaxcel capillary electrophoresis instrument and associated software which measured ratios of un-cleaved PCR product (wildtype or indels at target site) and cleaved PCR product (integration of BamHI target site through HDR) by quantifying the area of peaks for each of these different DNA species. [0095] Toxic ccdB Bacterial Screen: Chemically competent and ccdB-sensitive E. coli BW25141(λDE3) containing a ccdB toxic plasmid (under an arabinose-inducible promoter; previously described in Kleinstiver et al., Nature 2015; PMID: 26098369) with embedded zinc finger target sites were transformed plasmids encoding zinc finger-nuclease fusions and recovered in SOB media with 10 uM ZnCl for 60 mins, followed by addition of 10 mM IPTG and 60 more mins of recovery (total 2 hours). Transformations were plated on LB agar either containing chloramphenicol and 10 mM arabinose (selective media) or chloramphenicol (non-selective media). Cleavage of target site was estimated by dividing number of colonies on selective plates by number of colonies on non-selective plates.

### Example 5

### dCas9-AcuI and Zinc Finger-AcuI Fusions with Attenuated DNA Cleavage Kinetics

Mutations may be introduced to the AcuI nuclease domain to impact the nuclease activity of the AcuI fusions in order to introduce a nick at the target site, as well as to reduce potential off-targets of the platform. This has been demonstrated to be the case in FokI nuclease fusions to zinc fingers (Miller et al., Nat Biotech 2019; PMID: 31359006). Mutations that may attenuate AcuI cleavage kinetics are listed in Table 2 and encompass replacing a basic residue with a Serine and any Amidic residue with its acidic counterpart. Any combination of these mutations may also alter cleavage kinetics of AcuI to reduce off-targets or generate a nick at the target site.

TABLE 2
List of mutations to AcuI that modify the
nuclease activity of AcuI and AcuI fusions.
Single amino acid mutations to the nuclease domain
of AcuI that may lead to altered nuclease activity
of the enzyme and fusions to the AcuI domain.
AcuI Nuclease domain variant

H3S
H5S
K6S
K11S
R14S
N15D
N19D
R20S
K21S
N25D
R27S
N29D
R34S
K50S
N51D
K52S
K55S
N58D
R60S
K69S
H75S
K77S
K78S
R84S
R89S
K90S
K96S
K97S
H101S
N106D
K110S
Q111E
R113S
R114S
K120S
K122S
N128D
K140S
N148D
K149S
R151S
K153S
K154S
H156S
H163S
R173S
N180D
K183S
N190D
K191S
N193D
H194S
K203S
Q204E
N206D
R209S
K218S
Q220E
Q224E
N226D
N229D

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A DNA-binding domain (DBD) nuclease fusion protein comprising:
a) a dimerization-dependent nuclease domain, wherein the domain generates 3′ overhang double strand breaks in DNA; and
b) a DNA-binding domain (DBD),
wherein the dimerization-dependent nuclease domain is a Type IIS restriction enzyme nuclease domain, optionally an AcuI nuclease domain.

2. The fusion protein of claim 1, wherein the dimerization-dependent nuclease domain is linked to the DBD with an amino acid linker.

3. The fusion protein of claim 2, wherein the amino acid linker comprises the amino acid sequence of SEQ ID NO:2.

4. The fusion protein of claim 2, wherein the amino acid linker comprises the amino acid sequence of SEQ ID NO:3.

5. The fusion protein of claim 2, wherein the amino acid linker is an XTEN linker.

6. The fusion protein of claim 1, wherein the DBD is a zinc finger array.

7. The fusion protein of claim 1, wherein the DBD is a catalytically inactive Cas9 (dCas9) domain.

8. The fusion protein of claim 1, wherein the DBD is a TALE domain.

9. The fusion protein of claim 1, wherein the nuclease domain comprises an AcuI nuclease or an isoschizomer of AcuI nuclease.

10. The fusion protein of claim 9, wherein the nuclease domain is an AcuI nuclease that comprises an amino acid sequence that has at least 80%, at least 85%, at least 90%, or at least 95% sequence identity to the amino acid sequence of SEQ ID NO: 5.

11. The fusion protein of claim 10, wherein the amino acid domain is an AcuI nuclease domain that comprises an amino acid sequence that has at least 80%, at least 85%, at least 90%, or at least 95% sequence identity to the amino acid sequence of SEQ ID NO: 4.

12. The fusion protein of claim 11, wherein the AcuI nuclease domain contains H3S, H5S, K6S, K11S, R14S, N15D, N19D, R20S, K21S, N25D, R27S, N29D, R34S, K50S, N51D, K52S, K55S, N58D, R60S, K69S, H75S, K77S, K78S, R84S, R89S, K90S, K96S, K97S, H101S, N106D, K110S, Q111E, R113S, R114S, K120S, K122S, N128D, K140S, N148D, K149S, R151S, K153S, K154S, H156S, H163S, R173S, N180D, K183S, N190D, K191S, N193D, H194S, K203S, Q204E, N206D, R209S, K218S, Q220E, Q224E, N226D, or N229D substitution mutation, or any combination thereof.

13. The fusion protein of claim 9, wherein the nuclease domain is Eco57I nuclease.

14. The fusion protein of claim 1, wherein the nuclease domain is fused to an amino-terminal end of the DBD.

15. The fusion protein of claim 1, wherein the nuclease domain is fused to a carboxyl-terminal end of the DBD.

16. A DBD nuclease fusion protein dimer complex comprising two monomer fusion proteins, wherein each monomer is the fusion protein of claim 1.

17. The DBD nuclease fusion protein dimer complex of claim 16, wherein each of the DBD of the two monomer fusion proteins is a dCas9 domain, and the dimer complex binds to a target site in a PAM-out orientation.

18. A method of copying, incorporating, and/or inserting a nucleic acid sequence from an exogenous donor template into a nuclease target site of a genomic locus of a cell, the method comprising providing an exogenous donor template and a nucleic acid sequence encoding the DBD nuclease fusion protein of claim 1 to the nucleus of a cell,
wherein the exogenous donor template comprises sequences homologous to sequences within the nuclease target site of the genomic locus, and
wherein the DBD nuclease fusion protein binds to the nuclease target site and generates a 3′ overhang double strand break within the nuclease target site to induce homology-directed repair between the exogenous donor template sequences and the sequences surrounding the break,
thereby copying, incorporating, and/or inserting the nucleic acid sequence from the exogenous donor template into the nuclease target site of the genomic locus of the cell.

19. The method of claim 18, wherein the copied, incorporated, or inserted nucleic acid sequence replaces or corrects a mutated sequence within the nuclease target site of the genomic locus.

20. The method of claim 18, wherein the copied, incorporated, or inserted nucleic acid sequence inhibits expression of a gene within or adjacent to the nuclease target site of the genomic locus.

21. The method of claim 18, wherein the copied, incorporated, or inserted nucleic acid sequence activates expression of a gene within or adjacent to the nuclease target site of the genomic locus.

22. A method of copying, incorporating, and/or inserting a nucleic acid sequence from an exogenous donor template into a dCas9 target site of a genomic locus of a cell, the method comprising providing an exogenous donor template and a nucleic acid sequence encoding the dCas9 nuclease fusion protein of claim 7, and one or more dCas9-associated guide RNAs to the nucleus of a cell,
wherein the exogenous donor template comprises sequences homologous to sequences within the dCas9 target site of the genomic locus, and
wherein the dCas9 nuclease fusion protein forms a complex with one or more guide RNAs, and the complex binds to the dCas9 target site to generates a 3′ overhang double strand break within the dCas9 target site to induce homology-directed repair between the exogenous donor template sequences and the sequences surrounding the break,
thereby copying, incorporating, and/or inserting the nucleic acid sequence from the exogenous donor template into the dCas9 target site of the genomic locus of the cell.

23. The method of claim 22, wherein the copied, incorporated, or inserted heterologous nucleic acid sequence replaces or corrects a mutated sequence within the dCas9 target site of the genomic locus.

24. The method of claim 22, wherein the copied, incorporated, or inserted heterologous nucleic acid sequence inhibits expression of a gene within or adjacent to the dCas9 target site of the genomic locus.

25. The method of claim 22, wherein the copied, incorporated, or inserted heterologous nucleic acid sequence activates expression of a gene within or adjacent to the dCas9 target site of the genomic locus.

26. A method of copying, incorporating, and/or inserting a nucleic acid sequence from an exogenous donor template into a nuclease target site of a genomic locus of a cell, the method comprising providing an exogenous donor template and the zinc finger nuclease fusion protein of claim 6 to the nucleus of a cell,
wherein the exogenous donor template comprises sequences homologous to sequences within the nuclease target site of the genomic locus, and
wherein the zinc finger nuclease fusion protein binds to the nuclease target site and generates a 3′ overhang double strand break within the nuclease target site to induce homology-directed repair between the exogenous donor template sequences and the sequences surrounding the break,
thereby copying, incorporating, and/or inserting the nucleic acid sequence from the exogenous donor template into the nuclease target site of the genomic locus of the cell.

27. A method of copying, incorporating, and/or inserting a nucleic acid sequence from an exogenous donor template into a dCas9 target site of a genomic locus of a cell, the method comprising providing an exogenous donor template and dCas9 nuclease fusion protein of claim 7, and one or more dCas9-associated guide RNAs to the nucleus of a cell,
wherein the exogenous donor template comprises sequences homologous to sequences within the dCas9 target site of the genomic locus, and
wherein the dCas9 nuclease fusion protein is in a complex with one or more guide RNA(s), and the complex binds to the dCas9 target site and generates a 3′ overhang double strand break within the dCas9 target site to induce homology-directed repair between the exogenous donor template sequences and the sequences surrounding the break,
thereby copying, incorporating, and/or inserting the nucleic acid sequence from the exogenous donor template into the dCas9 target site of the genomic locus of the cell.

28. A method of copying, incorporating, and/or inserting a nucleic acid sequence from an exogenous donor template into a TALE target site of a genomic locus of a cell, the method comprising providing an exogenous donor template and TALE nuclease fusion protein of claim 8 to the nucleus of a cell,
wherein the exogenous donor template comprises sequences homologous to sequences within the TALE target site of the genomic locus, and
wherein the TALE nuclease fusion protein binds to the TALE target site and generates a 3′ overhang double strand break within the TALE target site to induce homology-directed repair between the exogenous donor template sequences and the sequences surrounding the break,
thereby copying, incorporating, and/or inserting the nucleic acid sequence from the exogenous donor template into the TALE target site of the genomic locus of the cell.

29. A method of introducing a variable-length insertion or deletion mutation that overlaps with a nuclease target site of a genomic locus of a cell, the method comprising providing the nucleic acid sequence encoding the zinc finger nuclease fusion protein of claim 6 to the nucleus of a cell,
wherein the zinc finger nuclease fusion protein binds to the nuclease target site and generates a 3′ overhang double strand break within the nuclease target site to induce repair of the break by non-homologous end-joining or microhomology-mediated end joining,
thereby leading to the generation of the variable-length insertion or deletion mutation that overlaps with the nuclease target site of the genomic locus of the cell.

30. A method of introducing a variable-length insertion or deletion mutation that overlaps with a TALE target site of a genomic locus of a cell, the method comprising providing the nucleic acid sequence encoding the TALE nuclease fusion protein of claim 8 to the nucleus of a cell,
wherein the TALE nuclease fusion protein binds to the TALE target site and generates a 3′ overhang double strand break within the TALE target site to induce repair of the break by non-homologous end-joining or microhomology-mediated end joining,
thereby leading to the generation of the variable-length insertion or deletion mutation that overlaps with the TALE target site of the genomic locus of the cell.

31. A method of introducing a variable-length insertion or deletion mutation that overlaps with a nuclease target site of a genomic locus of a cell, the method comprising:
a) providing the zinc finger nuclease fusion protein of claim 6 to the nucleus of a cell, wherein the zinc finger nuclease fusion protein binds to the nuclease target site and
b) generates a 3′ overhang double strand break within the nuclease target site to induce repair of the break by non-homologous end-joining or microhomology-mediated end joining,
thereby leading to the generation of the variable-length insertion or deletion mutation that overlaps the nuclease target site of the genomic locus of the cell.


SEQUENCE LISTING (13 sequences)

SEQ ID NO: 1 - protein, 8 aa, Artificial Sequence
  Zinc Finger Motif
  MISC_FEATURE (1)..(1): X can be any amino acid
  REPEAT (1)..(1): X can be present 2 times
  MISC_FEATURE (3)..(3): X can be any amino acid
  REPEAT (3)..(3): X can be present 2 or 4 times
  MISC_FEATURE (5)..(5): X can be any amino acid
  REPEAT (5)..(5): X can be present 12 times
  MISC_FEATURE (7)..(7): X can be any amino acid
  REPEAT (7)..(7): X can be present 3 to 5 times
XCXCXHXH

SEQ ID NO: 2 - protein, 4 aa, Artificial Sequence
  linker
LRGS

SEQ ID NO: 3 - protein, 5 aa, Artificial Sequence
  linker
GGGGS

SEQ ID NO: 4 - protein, 1000 aa, Artificial Sequence
  Acu1 restriction enzyme
MVHDHKLELAKLIRNYETNRKECLNSRYNETLLRSDYLDPFFELLGWDIKNKAGKPTNEREVVLEEALKASASEHSKKPDYTFRLFSERKFFLEAKKPSVHIESDNETAKQVRRYGFTAKLKISVLSNFEYLVIYDTSVKVDGDDTFNKARIKKYHYTEYETHFDEICDLLGRESVYSGNFDKEWLSIENKINHFSVDTLFLKQINTWRLLLGEEIYKYQPTIQENELNDIVQSYLNRIIFLRVCEDRNLETYQTLLNFASSNDFSALIDKFKQADRCYNSGLFDQLLTEQIIEDISSVFWVIIKQLYYPESPYSFSVFSSDILGNIYEIFLSEKLVINQSRVELVKKPENLDRDIVTTPTFIINDILRNTVLPKCYGKTDIEILQLKFADIACGSGAFLLELFQLLNDTLVDYYLSSDTSQLIPTGIGTYKLSYEIKRKVLLSCIFGIDKDLNAVEAAKFGLLLKLLEGEDVQSIANIRPVLPDLLDNILFGNSLLEPEKVELDHQVEVNPLDFSDLKFDVIVGNPPYMKSEDMKNITPLELPLYKKNYVSAYKQFDKYFLFLERGLALLKEEGILGYIVPSKFTKVGAGKKLRELLTDKGYLDSIVSFGANQIFQDKTTYTCLLILRKTPHTDFKYAEVRNLIDWKVRKADAMEFSSQQLSTLQSDAWILIPSELISVYHQILAQSQKLEDIVGIDNIFNGIQTSANDVYIFVPTHEDTENYYFIKKGQEYKIEKEITKPYFKTTSGEDNLYTYRTFKPNARVIYPYTQTESSVELIPLDEIREIFPLAYKYLMSLKFVLSSPKRDIKPRPKTTNEWHRYGRHQSLDNCGLSQKIIVGVLSVGDKYAIDTYGTLISSGGTAGYCVVALPDDCKYSIYYLQAILNSKYLEWFSALHGEVFRGGYIARGTKVLKNLPIRKIDFDNLEEANLHDLIATKQKELIEIYDKIDVNVNNKRVLTPLQRMFKREKEVLDQLLSRLYNLGVDDSLIPYIKDLYEAH

SEQ ID NO: 5 - protein, 199 aa, Artificial Sequence
  Putative endonuclease domain of Acu1
VHDHKLELAKLIRNYETNRKECLNSRYNETLLRSDYLDPFFELLGWDIKNKAGKPTNEREVVLEEALKASASEHSKKPDYTFRLFSERKFFLEAKKPSVHIESDNETAKQVRRYGFTAKLKISVLSNFEYLVIYDTSVKVDGDDTFNKARIKKYHYTEYETHFDEICDLLGRESVYSGNFDKEWLSIENKINHFSVDTL

SEQ ID NO: 6 - protein, 1262 aa, Artificial Sequence
  Alo1 restriction enzyme
MINQENLVALLNHLGFAKNKSVYSKSIGTTSLSVDIDKKEILYPEVDGFTVNERQICNFHANENFVVFECVHRLLEKGYNPEHIELEPKWKLGHGSSGGRADILIRDNFGKPLLLIECKNSGSEFNKSWSKTLQDGDQLFSYAQQISEIRFLCLYASDFYDSELVYQSNIIAHRDNEAYLVANPQFKNFKSATDVKERFSVWRDTYKLDFTTKGIFENNIQPYQIGKDKYSIADLHAIAASDQQKKYHQFATILRQHNVSGRENAFDKLVNLFLCKLVDEIENPNDLKFYWKGVAYDSHFDLMDRLQQLYQSGMDKFLGEDITYINQNDVTNALRFIRQKPDATHRAVWNLFVKQKFFTNNDFSFLDVHNERLFYQNAEVLLKVLQMWQDIRLTSATGHNQFLGDMFEGFLDQGVKQSEGQFFTPMPICRFILMSLPLESLVRDNPTPPMAVDYACGAGHFLTELALQFQPLLEQHKPLAAPAEYHKSMVGIEKEYRLSKVAKVSAFMYGHQGIQVCYGDGLVNSHEAFPDIRDGHFDLLVANPPYSVRGFLETLPEEDRKAYSLTNTINDTETANSIETFFIERAKQLLKSGGVAAIILPASILSNGGSAYIRAREILLQYFDIVAIAEFGSGTFGKTGTNTVSLFLRRKRTQPDTAEHYRERIEEWFKSCTTSKRKQVLYKDGHLIEKYCAHINVPLADYQSFLRGEAEGSWMSHEHFQSYHDKFDTSTELANLRKQRKFKALSEYEQTAEIAKRYLGYVHSIERDKLYHFCLASDQTNPVLIIRSPSGTKEMKQFLGYEWSSAKGDEGIKLIEDTSGKHVTKLYDGPNHANPTLFNRANPTKLNSYIAANFEGTLGKISPEVKDLTNVVQLVDMLDLKRSTFDKQLSLVAKKSVHIASKWPQVKVGSICSFEYGKPLPEENRVSGPYPVMGSNGRVGYHSEYLIKGPAIIIGRKGSAGQVVWEEEDCYPIDTTFYAKTLTSDIDKYFLFHVLKELDLGHLQGGVGVPGLNRNEAHELPMPLPPIKVQEQMVVDFKKIDADVASAAALVSDSLSRINSEVDSLYSSGVGRISIEEISTNVQYGLNEKMNETGIGYKTFRMNEVIDGRMVDNGKMKRANISAKEFSKYQLNKGDLLFIRSNGSLEHIGRFGLFDLDGEYCYASYLVRIVADTSKIRPYYLAIIMNSAALRKEVVSLAVKSGGTNNINATKMKSIKVPVPSLDEQAKFIAKIELLQKQVADAQATIDSAAARKSTVMKKYLL

SEQ ID NO: 7 - protein, 199 aa, Artificial Sequence
  Putative endonuclease domain of Alo1
INQENLVALLNHLGFAKNKSVYSKSIGTTSLSVDIDKKEILYPEVDGFTVNERQICNFHANENFVVFECVHRLLEKGYNPEHIELEPKWKLGHGSSGGRADILIRDNFGKPLLLIECKNSGSEFNKSWSKTLQDGDQLFSYAQQISEIRFLCLYASDFYDSELVYQSNIIAHRDNEAYLVANPQFKNFKSATDVKERFS

SEQ ID NO: 8 - protein, 1009 aa, Artificial Sequence
  Bpm1 restriction enzyme
MHISELVDKYKAHRSTFLKPTYNETQLRNDFIDPLLKSLGWDVDNTKGKTHILRDVIQEEYIEIKDEETKKNPDYTLRINGTRKLFVEVKKPSFNILKSAKAAFQTRRYGWSANLGISVLTNFEHLVIYDCRYTPDKSDNEHIARYKVFSYEEYEEAFDEIKDIISYESANSGALDEMFDVNTRVGETFDEYFLQQIENWREKLAKTAIKNNTELGEEDVNFIVQRLLNRIIFLRVCEDRTIEKYETIKSIKNYEELKDLFQKSDRKFNSGLFDFIDDTLLLEVEIDSNVLIEIFSDLYFPQSPYDFSVVDPTILSQIYERFLGQEIIIESGGTFHITESPEVAASNGVVPTPKIIVEQIVKDTLTPLTEGKKFNELCNLKIADICCGSGTFLISSYDFLVEKVMEKIIEENIDDSDLVYETEEGLILTLKAKRNILENNLFGVDVNPYAVEVAEFSLLLKLLEGENEASVNNFIHEHEDKILPDLTSIIKCGNSLVDNKFFEFMPESLEDDEILFKANPFEWEEEFPDIMANGGFDAIIGNPPYVRIQNMKKYSPEEIEYYQSKDSEYTVAKKETVDKYFLFIERALILLNPTGLLGYIIPHKFFITKGGKELRKFIAEKHQISKIINFGVTQVFPGRATYTAILIIQANKMAQFKYKKVSNISAETLDSEENTCVYSSEKYNSDPWIFLSPETEAVFTKFTEAQFEKLGEITDISVGLQTSADKIYIFIPENETSDTYIFNYKGKRYEIEKSICCPAIYDLSFGSFESIQGNAQMIFPYEIRDEEAYLLEEETLENDYPLAWNYLNEFKEALEKRSLQGRNPKWYQYGRSQSLSKFHDKEKLIWTVLATKPPYVLDRNNLLFTGGGNGPYYGLINQSIYSLHYFLGILSHPVIESMVKARASEFRGSYYSHGKQFIEKIPIRKIDFDDQDEVDKYNTVVTTVEKLIITTDRIKSESNGPRRRMLRRRLDALSNQLIQVINELYNISDEEYTTVLNDEMLTAALGEEK

SEQ ID NO: 9 - protein, 199 aa, Artificial Sequence
  Putative endonuclease domain of Bpm1
HISELVDKYKAHRSTFLKPTYNETQLRNDFIDPLLKSLGWDVDNTKGKTHILRDVIQEEYIEIKDEETKKNPDYTLRINGTRKLFVEVKKPSFNILKSAKAAFQTRRYGWSANLGISVLTNFEHLVIYDCRYTPDKSDNEHIARYKVFSYEEYEEAFDEIKDIISYESANSGALDEMFDVNTRVGETFDEYFLQQIENW

SEQ ID NO: 10 - protein, 529 aa, Artificial Sequence
  Bae1 restriction enzyme
MGRAASLLKAHWRGAFFSEAGALGAPSSWPVLADRAHEPELADVAAAADSGRPVALPHSCVELTLDDAGVAVARISDPATRNAFTPALVAAMEAVVAWASETPGCKVLILTGHGHYFATGGTREGLQAIQQGNAKFTDARLYELPLACPVPVIAAMQGHAIGAGWAMGMACDAVLFAEESVYHSPYLSYGFTPGAGSTLVFPMRLGLDLGREILLGARPYKGRELRERLPGLSVAPRGEVLARARRIAAQWSAQRTRNELIRDKRQALAALAQAMPAAIRKELAMHEQTFHHNPDVARRIDRAYGVGATPAADMPSTPARDLAALLKSTLAAEIQLSADELDDEAGFVDLGLDSVTAVTWARTLGRALSIELTPAHVYQYPSVAKLLAYLREATDGVAVRGDVGGADAVPAAAPAPGPKPQPTLEPTPAAAAGIAIVEIVRQTLAAELQLDAQDIDAHASFVDMGLDSVTAVTWARKLHERLAVDLSPTQLYQHPNVAALCAHLSDAGASAGPDAAAHAAADRAARTAA

SEQ ID NO: 11 - protein, 193 aa, Artificial Sequence
  Putative endonuclease domain of Bae1
MGRAASLLKAHWRGAFFSEAGALGAPSSWPVLADRAHEPELADVAAAADSGRPVALPHSCVELTLDDAGVAVARISDPATRNAFTPALVAAMEAVVAWASETPGCKVLILTGHGHYFATGGTREGLQAIQQGNAKFTDARLYELPLACPVPVIAAMQGHAIGAGWAMGMACDAVLFAEESVYHSPYLSYGFTP

SEQ ID NO: 12 - protein, 919 aa, Artificial Sequence
  Mme1 restriction enzyme
MALSWNEIRRKAIEFSKRWEDASDENSQAKPFLIDFFEVFGITNKRVATFEHAVKKFAKAHKEQSRGFVDLFWPGILLIEMKSRGKDLDKAYDQALDYFSGIAERDLPRYVLVCDFQRFRLTDLITKESVEFLLKDLYQNVRSFGFIAGYQTQVIKPQDPINIKAAERMGKLHDTLKLVGYEGHALELYLVRLLFCLFAEDTTIFEKSLFQEYIETKTLEDGSDLAHHINTLFYVLNTPEQKRLKNLDEHLAAFPYINGKLFEEPLPPAQFDKAMREALLDLCSLDWSRISPAIFGSLFQSIMDAKKRRNLGAHYTSEANILKLIKPLFLDELWVEFEKVKNNKNKLLAFHKKLRGLTFFDPACGCGNFLVITYRELRLLEIEVLRGLHRGGQQVLDIEHLIQINVDQFFGIEIEEFPAQIAQVALWLTDHQMNMKISDEFGNYFARIPLKSTPHILNANALQIDWNDVLEAKKCCFILGNPPFVGKSKQTPGQKADLLSVFGNLKSASDLDLVAAWYPKAAHYIQTNANIRCAFVSTNSITQGEQVSLLWPLLLSLGIKINFAHRTFSWTNEASGVAAVHCVIIGFGLKDSDEKIIYEYESINGEPLAIKAKNINPYLRDGVDVIACKRQQPISKLPSMRYGNKPTDDGNFLFTDEEKNQFITNEPSSEKYFRRFVGGDEFINNTSRWCLWLDGADISEIRAMPLVLARIKKVQEFRLKSSAKPTRQSASTPMKFFYISQPDTDYLLIPETSSENRQFIPIGFVDRNVISSNATYHIPSAEPLIFGLLSSTMHNCWMRNVGGRLESRYRYSASLVYNTFPWIQPNEKQSKAIEEAAFAILKARSNYPNESLAGLYDPKTMPSELLKAHQKLDKAVDSVYGFKGPNTEIARIAFLFETYQKMTSLLPPEKEIKKSKGKN

SEQ ID NO: 13 - protein, 155 aa, Artificial Sequence
  Putative endonuclease domain of Mme1
MALSWNEIRRKAIEFSKRWEDASDENSQAKPFLIDFFEVFGITNKRVATFEHAVKKFAKAHKEQSRGFVDLFWPGILLIEMKSRGKDLDKAYDQALDYFSGIAERDLPRYVLVCDFQRFRLTDLITKESVEFLLKDLYQNVRSFGFIAGYQTQVI